SEQUENCE LISTING (30 sequences)

SEQ ID NO: 1 - protein, 14 aa, synthetic construct [sequence omitted]

SEQ ID NO: 2 - protein, 9 aa, synthetic construct
  DISULFID 1..9: note = disulfide bridge
CRGDKGPDC

SEQ ID NO: 3 - protein, 17 aa, synthetic construct
  SITE 1..4: note = D-Glutamic acid
  SITE 7: note = D-Asparagine
  SITE 8: note = D-Aspartic acid
  SITE 9: note = D-Asparagine
  SITE 10..11: note = D-Glutamic acid
  SITE 15: note = D-Serine
  SITE 17: note = D-Arginine
EEEEGVNDNEEGFFSAR

SEQ ID NO: 4 - protein, 8 aa, synthetic construct
  SITE 1..8: note = D-Glutamic acid
EEEEEEEE

SEQ ID NO: 5 - protein, 9 aa, synthetic construct
  SITE 1..9: note = D-Glutamic acid
EEEEEEEEE

SEQ ID NO: 6 - protein, 16 aa, synthetic construct
  SITE 1..2: note = D-Lysine
  SITE 6..7: note = D-Lysine
  SITE 11..12: note = D-Lysine
  SITE 16: note = D-Lysine
  SITE 3..5: note = D-Glutamic acid
  SITE 8..10: note = D-Glutamic acid
  SITE 13..15: note = D-Glutamic acid
KKEEEKKEEEKKEEEK

SEQ ID NO: 7 - protein, 10 aa, synthetic construct
  SITE 1: note = D-Glutamic acid
  SITE 3: note = D-Glutamic acid
  SITE 5: note = D-Glutamic acid
  SITE 7: note = D-Glutamic acid
  SITE 9: note = D-Glutamic acid
  SITE 2: note = D-Lysine
  SITE 4: note = D-Lysine
  SITE 6: note = D-Lysine
  SITE 8: note = D-Lysine
  SITE 10: note = D-Lysine
EKEKEKEKEK

SEQ ID NO: 8 - protein, 10 aa, synthetic construct
GGPLGVRGKC

SEQ ID NO: 9 - DNA, 10 bp, synthetic construct
  source 1..10: mol_type = other DNA
  modified_base 1..10: mod_base = OTHER; note = phosphorothioate backbone nucleosides
tcatagttag

SEQ ID NO: 10 - skipped

SEQ ID NO: 11 - protein, 5 aa, synthetic construct
LGGGA

SEQ ID NO: 12 - protein, 10 aa, synthetic construct
KITAQDDEES

SEQ ID NO: 13 - protein, 10 aa, synthetic construct
GGLVPRGSGC

SEQ ID NO: 14 - protein, 11 aa, synthetic construct
GGPVPLSLVMC

SEQ ID NO: 15 - protein, 13 aa, synthetic construct
GGGSGRSANAKGC

SEQ ID NO: 16 - protein, 8 aa, synthetic construct
GGAAFAGC

SEQ ID NO: 17 - protein, 9 aa, synthetic construct
  SITE 3: note = D-Phenylalanine
GGFPRSGGC

SEQ ID NO: 18 - protein, 11 aa, synthetic construct
GNSGRAVTYGC

SEQ ID NO: 19 - protein, 11 aa, synthetic construct
GKPISLISSGC

SEQ ID NO: 20 - protein, 12 aa, synthetic construct
GRPLALWESQGC

SEQ ID NO: 21 - protein, 10 aa, synthetic construct
GGAIEFDSGC

SEQ ID NO: 22 - protein, 10 aa, synthetic construct
GPMKRLTLGC

SEQ ID NO: 23 - protein, 12 aa, synthetic construct
  DISULFID 4..12: note = disulfide bridge
CGGCRGDKGPDC

SEQ ID NO: 24 - protein, 9 aa, synthetic construct
CGNKRTRGC

SEQ ID NO: 25 - protein, 7 aa, synthetic construct
RGDKGPD

SEQ ID NO: 26 - protein, 11 aa, synthetic construct
GGCRGDKGPDC

SEQ ID NO: 27 - DNA, 10 bp, synthetic construct
  source 1..10: mol_type = other DNA
  misc_feature 10: note = Thiol
tcatagttag

SEQ ID NO: 28 - protein, 24 aa, synthetic construct
  MOD_RES 6: note = Phosphorylated tyrosine
  MOD_RES 19: note = Phosphorylated tyrosine
GPGGDYAAMGACPASEQGYEEMRA

SEQ ID NO: 29 - protein, 15 aa, synthetic construct
  SITE 8..15: note = D-arginine
ALAPYIPRRRRRRRR

SEQ ID NO: 30 - protein, 7 aa, synthetic construct
ALAPYIP

(19) United States
(12) Patent Application Publication
(10) Pub. No.: US 2026/0315619 A1
(43) Pub. Date: Oct. 15, 2026

(54) REAGENTS AND METHODS FOR THE CONDITIONAL DELIVERY OF CARGO

(71) Applicant: Massachusetts Institute of Technology, Cambridge, MA (US)

(72) Inventors: Sangeeta N. Bhatia, Lexington, MA (US); Chayanon Ngambenjawong, Cambridge, MA (US); Edward Tan, Cambridge, MA (US); Qian Zhong, Boston, MA (US)

(73) Assignee: Massachusetts Institute of Technology, Cambridge, MA (US)

(21) Appl. No.: 19/120,088

(22) PCT Filed: Oct. 10, 2023
(86) PCT No.: PCT/US2023/076467

§ 371 Date: Apr. 10, 2025

Related U.S. Application Data
(60) Provisional application No. 63/415,002, filed on Oct. 11, 2022.

(51) Int. Cl. A61K 47/69; A61K 47/60; A61K 47/65

(52) U.S. Cl. CPC A61K 47/6911; A61K 47/60; A61K 47/65

(57) ABSTRACT

Aspects of the disclosure relate to reagents, compositions, kits, and methods associated with liposome-mediated delivers7 of cargo in response to an environmental trigger. Some aspects of the present invention relate to treating a diseased cell, tissue, or organ using the reagents.

### RELATED APPLICATIONS

This application claims the benefit under 35 U.S.C. § 119(e) of the U.S. provisional application No. 63/415,002, filed Oct. 11, 2022, which is incorporated by reference herein in its entirety.

### REFERENCE TO AN ELECTRONIC SEQUENCE LISTING

The contents of the electronic sequence listing (M065670525WO00-SEQ-FL.xml; Size: 42,016 bytes; and Date of Creation: Oct. 10, 2023) is herein incorporated by reference in its entirety.

### BACKGROUND OF THE INVENTION

Effective and precise delivery of therapeutics to their targets on disease sites are useful in treating infections and a range of non-communicable conditions, including cancer, chronic respiratory diseases, and cardiovascular disorders. Therapeutics directed at intracellular targets are rapidly being developed. Such targets include antibodies, enzymes, peptides, nucleic acids, intrabodies, and ribonucleoprotein particles (RNPs). To promote precision medicine, efficient and targeted methods of delivering cargo to cells are needed.

### SUMMARY OF THE INVENTION

Aspects of the present disclosure provide reagents comprising: (i) a liposome comprising a cationic lipid that is positively charged at a pH less than 9; (ii) a fusion inhibitor linked to the liposome via (iii) an environmentally-responsive linker, wherein the fusion inhibitor comprises:
(a) nucleic acid, protein, and/or synthetic polymer, optionally wherein the nucleic acid has an overall negative net charge, optionally wherein the nucleic acid fusion inhibitor is 5-30 nucleotides in length, optionally comprising one or more modified nucleotides;
(b) a peptide comprising one or more negatively charged amino acids; and/or
(c) a zwitterionic molecule,
and wherein the liposome is positively charged in the absence of the fusion inhibitor, optionally wherein the environmentally-responsive linker is a protease substrate, optionally wherein the liposome comprises a lipid linked to the fusion inhibitor, optionally wherein the protein is an antibody, and optionally wherein the fusion inhibitor comprises (b) and/or (c).

In some embodiments, a reagent comprises one or more lipids selected from the group consisting of 1,2-dioleoyl-3-trimethylammonium-propane (DOTAP), 1,2-dioleoyl-3-dimethylammonium propane (DODAP), 1,2-dimyristoyl-sn-glycero-3-phosphocholine (DMPC), 1,2-dimyristoyl-sn-glycero-3-phosphoethanolamine (DMPE), cholesterol, 1,2-dioleoyl-sn-glycero-3-phosphoethanolamine (DOPE), 1,2-dioleoyl-sn-glycero-3-phosphoethanolamine maleimide (DOPE-MAL), 1,2-distearoyl-sn-glycero-3-phosphoethanolamine maleimide (DSPE-MAL), 1,2-distearoyl-sn-glycero-3-phosphoethanolamine-N-[amino (polyethylene glycol)-2000] maleimide (DSPE-PEG2k-MAL), 1,2-distearoyl-sn-glycero-3-phosphoethanolamine-N-[amino (polyethylene glycol)-2000] DBCO (DSPE-PEG2k-DBCO), 1,2-dimyristoyl-rac-glycero-3-methoxypolyethylene glycol-2000 maleimide (DMG-PEG2k-MAL), C14PEG1k, C14PEG2k, DSPE-PEG2K, DMG-PEG1k, and DMG-PEK2k.

In some embodiments, the reagent comprises a ligand capable of binding to a cell.

In some embodiments, the ligand comprises a peptide, ligand, lectin, antibody, and/or nucleic acid molecule, optionally wherein the ligand is a tumor-penetrating ligand and optionally wherein the antibody is a nanobody.

In some embodiments, the ligand is an iRGD peptide, optionally wherein the iRGD peptide comprises the amino acid sequence CRGDKGPDC (SEQ ID NO: 2), in which the two cysteines in SEQ ID NO: 2 form a disulfide bridge.

In some embodiments, the fusion inhibitor comprises a glutamate residue and/or the protease substrate is a cell-specific substrate, tissue-specific substrate, organ-specific substrate, and/or a disease-specific substrate.

In some embodiments, the fusion inhibitor comprises overall net charge of inhibitor peptides is −3 at physiological pH, optionally wherein the physiological pH is pH 7.4 and optionally wherein the fusion inhibitor comprises at least three negatively charged amino acids.

In some embodiments, the fusion inhibitor is the peptide of (b) and less than 25% of the peptide is hydrophobic amino acid residues and/or the zwitterionic molecule is a peptide.

In some embodiments, the fusion inhibitor comprises a lysine or arginine residue.

In some embodiments, the liposome comprises more than 5% but less than 50% DOTAP.

In some embodiments, the fusion inhibitor is 5-25 amino acids in length, optionally wherein the fusion inhibitor is 8-17 amino acids in length.

In some embodiments, the reagent comprises: DOTAP, DMPC, DOPE-MAL, and DSPE-PEG2k-MAL.

In some embodiments, the ratio of DOTAP/DMPC/DOPE-MAL/DSPE-PEG2k-MAL is 20/70/7/3.

In some embodiments, the peptide comprises the amino acid sequence:

(a)
(SEQ ID NO: 1)
eGVndneeGFFsAr;
(b)
(SEQ ID NO: 3)
eeeeGVndneeGFFsAr;
(c)
(SEQ ID NO: 4)
eeeeeeee;
(d)
(SEQ ID NO: 5)
eeeeeeeee;
(e)
(SEQ ID NO: 6)
kkeeekkeeekkeeck;
and/or
(f)
(SEQ ID NO: 7)
ekekekekek,

wherein a lower case letter in an amino acid sequence indicates a D-isomer amino acid.

Further aspects of the composition comprises any of the reagents of the disclosure, wherein the liposome encapsulates cargo, optionally wherein the cargo is a therapeutic molecule, optionally wherein the fusion inhibitor is a therapeutic molecule, optionally wherein the therapeutic molecule that is the fusion inhibitor and/or the cargo is a therapeutic nucleic acid, a therapeutic peptide, or a therapeutic protein, optionally wherein the cargo is a molecule that is greater than 1 kDa in size.

In some embodiments, the composition further comprises a pharmaceutically acceptable excipient, optionally wherein the pharmaceutically acceptable excipient is sucrose or lactose, optionally wherein the composition comprises the reagent in a solution comprising the pharmaceutically acceptable excipient, optionally wherein the solution comprises between 5% to 25% weight per volume (w/v) of the pharmaceutically acceptable excipient.

In some embodiments, the therapeutic molecule is capable of inducing cell death in a target cell and/or wherein the therapeutic molecule is granzyme B or phosphorylated PI3K proteolysis targeting chimeric molecule (pPROTAC).

Further aspects of the present disclosure provide methods comprising administering any of the compositions of the disclosure to a subject, optionally wherein the method comprises detecting the fusion inhibitor in a sample from the subject, optionally wherein the sample is urine, optionally wherein the fusion inhibitor targets the reagent to a cell, tissue, and/or organ in the subject.

In some embodiments, (a) the subject has cancer, optionally wherein the cancer is lung cancer; (b) the reagent localizes to a cell, tissue, and/or organ more than another cell, tissue, and/or organ; (c) delivery of the cargo to a cell, tissue, or organ expressing a protease is increased relative to a cell, tissue, and/or organ with reduced expression of the protease; and/or (d) the method delivers a therapeutic molecule capable of inducing cell death in a target cell.

In some embodiments, the method reduces the size of a tumor in the subject.

Each of the embodiments of the invention can encompass various recitations made herein. It is, therefore, anticipated that each of the recitations of the invention involving any one element or combinations of elements can, optionally, be included in each aspect of the invention.

### BRIEF DESCRIPTIONS OF THE DRAWINGS

FIG. 1 depicts a schematic showing conditional cytosolic delivery of granzyme B using synthetic T cells at a target cell membrane.

FIGS. 2A-2E show in vitro characterization of CFLIPs. FIG. 2A shows representative cryo-TEM images of neutrophil elastase (NE)-activatable CFLIPs before (left) and after (right) incubation with recombinant NE. The diameter of the liposomes before and after activation averaged 54±9 nm and 55±14 nm, respectively. FIG. 2B shows graphs representing the size and polydispersity of original FLIPs, CFLIPs, and activated CFLIPs (i.e., CFLIPs+proteases) via dynamic light scattering (DLS). The FLIPs differed slightly in size and polydispersity when functionalized on the surface with negatively charged peptides via substrate linkers in response to a representative serine protease (NE) or metalloprotease (MMP9). Abbreviations: fusogenic liposomes (FLIPs), conditional fusogenic liposomes (CFLIPs), neutrophil elastase (NE), and matrix metallopeptidase 9 (MMP9). FIG. 2C shows measurements of the zeta potentials of original FLIPs, CFLIPs, and activated CFLIPs. Negatively charged peptide fusion inhibitors on the surface turned the zeta potential of FLIPs negative, and the surface charge was restored upon the protease cleavage of the respective substrate linker. FIG. 2D shows confocal microscopy images demonstrating fluorescently labeled FLIPs (Dil) marked the cell membrane after 1 hour of co-incubation, while non-fusogenic liposomes with either positive (N-FLIP1) or negative (N-FLIP2) charges on the surface were mainly located in the lysosomes in the perinuclear area. FIG. 2E shows confocal microscopy images demonstrating the conjugation of peptide-based fusion inhibitors to the FLIPs (NE−; forming CFLIPs) significantly suppresses the interaction between cancer cells and liposomes (both fusion with the cells and endocytosis). Upon in vitro activation by recombinant NE (NE+; i.e., fusion inhibitors were cleaved), the liposomes regained fusogenic properties as indicated. Nuclei were stained with Hoechst 33342 and lysosomes were stained with LysoTracker Green DND-26 in FIGS. 2D and 2E.

FIGS. 3A-3D show confocal microscopy images demonstrating that increasing cationic lipid ratios facilitate fusion with cellular plasma membranes. Dil alone or liposomal formulations labeled with Dil were incubated with mouse KP lung cancer cells for 15 minutes prior to washing. The cells were imaged at 15 minutes, then imaged again 4 hours later. FIG. 3A demonstrates that lipophilic Dil rapidly labeled cellular plasma membranes and endolysosomes (15-minute incubation). The accumulation of Dil in endosomes and lysosomes increased over time (4 hours after the removal of Dil bottom row). Liposomes with 10% of the cationic lipid DOTAP in total lipids (by molar ratio) (FIG. 3B; FLIP10, +3 mV) showed negligible fusion with the cellular plasma membrane in comparison to those with 20% (FIG. 3C; FLIP20, +14 mV) and 50% (FIG. 3D; FLIP50, +17 mV) DOTAP at both timepoints.

FIGS. 4A-4D show confocal microscopy images of cells incubated with CFLIPs formulated with a panel of fusion inhibitors. FLIP20 liposomes were conjugated to various potential fusion inhibitors, including peptide P3 (FIG. 4A; FLIP20-P3, +14 mV), peptides P3 and iRGD (FIG. 4B; FLIP20-P3-iRGD, +15 mV), peptide 17 (FIG. 4C, FLIP20-(EK)₅, +18 mV) and the synthetic polymer PEG2k (FIG. 4D; FLIP20-PEG2k, +8 mV). See Table 7 for inhibitor compositions. The indicated formulations were incubated with mouse KP lung cells for 15 minutes prior to washing. The cells were imaged at 15 minutes, then imaged again 4 hours later.

FIGS. 5A-5D show CFLIPs directly deliver cargo into the cytosol in a membrane fusion-mediated, perforin-free manner. FIG. 5A shows a representative cryo-TEM image of protease-activatable CFLIPs with mouse granzyme B (mGzmB) encapsulated in the aqueous core. The size of the mGzmB-encapsulated CFLIPs averaged 65±4 nm in diameter. Confocal microscopy revealed that Cy5-labeled GzmB (FIG. 5B) and calcein (FIG. 5C) delivered by pre-activated CFLIPs was widely spread throughout the cytoplasm (upper rows), while the cargo delivered via intact CFLIPs was mainly colocalized with lysosomal compartments (bottom rows). Nuclei were stained with Hoechst 33342: lysosomes were stained with LysoTracker Red DND-26 or LysoTracker Deep Red. Calcein staining and Cy5-labeled GzmB staining are also shown. Confocal microscopy was used to image nuclei, endolysosomes, and delivered cargo calcein in cytoplasm. FIG. 5D shows confocal microscopy fluorescence images of Cy5-tagged bovine serum albumin (BSA) delivered to the cytosol via MMP9 activatable CFLIPs.

FIGS. 6A-6E show mouse GzmB-loaded CFLIPs have strong in vivo antitumor potency via caspase 3-mediated programmed cell death. FIG. 6A is a graph showing the catalytic activity of GzmB after multiple freeze-thaw cycles and sonication. Multiple freeze-thaw cycles did not compromise GzmB activity (FTC10=10 freeze-thaw cycles between dry ice and at 37° C.), while 3 minutes of sonication in a bath sonicator reduced GzmB activity by approximately 30%. FRET-paired (Cy5 and its quencher QSY21) granzyme B peptide substrates were incubated with the granzyme B (treated with various conditions) to measure cleaving activity (i.e. catalytic activity). Samples without GzmB (i.e., GzmB−) were used as a negative reference (hollow square). Fluorescence activation (de-quenching) was recorded for 45 min. Förster resonance energy transfer=FRET. FIG. 6B is a graph showing the in vitro toxicity of GzmB-loaded CFLIPs (CFLIP/GzmB) on mouse KP lung cancer cells. Incubation of the cells with of naked GzmB, empty CFLIP, and CFLIP/GzmB was performed for 48 hours, and viable cells were measured with an MTS assay. FIG. 6C shows a graph depicting levels of active caspase 3 in lysates from cells treated with the different FLIP formulations in FIG. 6B. Active caspase 3 was only detected in the cell lysates treated with the pre-activated CFLIP/GzmB. A bioluminescence-based substrate test was used to detect activation of pro-caspase 3 by GzmB. FIG. 6D shows an immunoblot further confirming the presence of active caspase 3 (19 kD and 17 kD fragments) only in the CFLIP/GzmB pre-activated with MMP9. Abbreviations: CFLIP empty vehicle=empty CFLIP with no GzmB encapsulated, but not activated by MMP9; CFLIP/GzmB=CFLIP with GzmB encapsulated and activated by MMP9. FIG. 6E shows the viability of multiple cell lines after incubation with CFLIP/GzmB. Incubation of the cells with CFLIP/GzmB was performed for 48 hours, and viable cells were measured with an MTS assay. CFLIP/GzmB showed broad potency against multiple mouse cancer cell lines. B16F10=mouse melanoma; 4T1=mouse breast cancer; MC38=mouse colon cancer; KPM=metastatic mouse lung KP cells.

FIGS. 7A-7C show the in vitro delivery of phosphor-PROTAC into the cell cytosol via CFLIP. FIG. 7A shows a schematic diagram of conditional post-translational target protein knockdown by a phosphorylated PROTAC. Upon activation of the RTK by extracellular growth factors, the PROTAC is phosphorylated, creating a binding site for the SH2- or PTB-domain-containing effector protein and its subsequent recruitment for ubiquitination by VHL and proteasomal degradation (FIG. 7A was adapted from Reference 11). FIG. 7B shows the peptide sequence of the phosphor-PROTAC (pPI3K) with and without a cell-penetrating peptide (CPP). The PROTAC peptide comprises three domains: 1) a p85-binder, 2) a polyethylene oxide oligomer (PEO) linker, and 3) an E3 ligase binder. Phosphorylation occurs at two labeled tyrosine residues (pY) on the peptide ligand, which enables the peptide, once in the cytosol, to bind to the p85 catalytic domain of PI3K without the activation of RTK. Abbreviations: D-R=D-isomer of arginine (FIG. 7B was adapted from Reference 11). FIG. 7C shows an immunoblot of lysates from cells treated with phosphor-PROTAC administered through CPP (left) or pre-activated CFLIP (right). PI3K and its downstream signaling molecule (pAkt) were degraded when using pPI3K-PROTAC delivered with pre-activated CFLIPs (total Akt levels unchanged). Conversely, CPP-modified pPI3K-PROTAC failed to degrade its protein target (i.e., p85) and downstream pAkt. Beta-Actin was chosen as a reference protein to ensure the same amount of protein was loaded into each lane.

FIGS. 8A-8E demonstrate the pharmacokinetics, biodistribution, and minimal systemic toxicity of CFLIPs. FIG. 8A shows the pharmacokinetics of the original FLIPs and CFLIPs. CFLIP (IRGD+)=MMP9-activatable CFLIP conjugated to an iRGD peptide as a tumor-targeting ligand; CFLIP (IRGD−)=MMP9-activatable CFLIP without iRGD. FLIPs/CFLIPs were fluorescently tagged with DiR. Liposomal DiR in circulation was tracked and quantified for the fluorescence at different plasma sampling time with LI-Cor IVIS. The DiR-liposomes sampled at 1 min were set as 100% of injected dose (assuming all injected liposomes are circulating in the blood). FIG. 8B shows the increased CFLIP liposomal accumulation in lung tumors due to tumor-specific protease cleavage and homing peptide targeting. LI-COR IVIS imaging was used. Liposomal DiR in tissues 24 h after administration were imaged and quantified for the fluorescence at 24 h after administration with LI-Cor IVIS. FIG. 8C depicts the timeline for the efficacy study in a KP lung tumor model. 500,000 luciferase-expressing mouse KP lung cancer cells were injected intravenously via tail vein. Treatments were administered on Days 7, 9, 11, 14, and 16 for five total doses. Tumor burdens were regularly monitored via IVIS imaging for luciferin bioluminescence. FIG. 8D shows a graph monitoring the progression of tumor growth as measured via IVIS. Mice treated with synthetic T cells showed significant tumor growth suppression with each dose escalating to 3 mg GzmB/kg body weight. FIG. 8E shows the body weight of mice treated with 3 mg GzmB/kg body of synthetic T cells for 5 doses, demonstrating no observed acute toxicity in a mouse model with KP lung tumors.

FIGS. 9A-9G show the in vivo performance of CFLIPs as anti-tumor treatment. FIG. 9A depicts the timeline for the efficacy study in a KP lung tumor model. 500,000 luciferase-expressing mouse KP lung cancer cells were injected intravenously via tail vein. Treatments were administered on Days 7, 9, 11, 14, and 16 for five total doses. Tumor burdens were regularly monitored via IVIS imaging for luciferin bioluminescence. FIG. 9B shows a graph monitoring the progression of tumor growth in KP tumor-bearing mice treated as indicated, measured via IVIS. The GzmB-encapsulated CFLIP (synthetic T cells) showed moderate tumor growth suppression when used as a monotherapy in the KP tumor-bearing mice. The animal cohorts were treated with: PBS (IV injection); GzmB (IV injection); N-FLIP/GzmB (non-cleavable FLIP with GzmB encapsulated, IV injection); or CFLIP/GzmB (activatable synthetic T cells, IV injection). FIG. 9C shows a graph monitoring the progression of tumor growth in KP tumor-bearing mice treated as indicated, measured via IVIS. The animal cohorts were treated with: PBS (IV injection), anti-PD1 immune checkpoint inhibitor (PD-1, i.p. injection); synthetic T cells (CFLIP/GzmB, IV injection), synthetic T cells in combination with an anti-PD1 immune checkpoint inhibitor (CFLIP/mGzmB/PD-1). Abbreviations: IV=intravenous; i.p.=intraperitoneal. Treatments were administered at times indicated by arrows. Treatment with the immune checkpoint blockade anti-PD1 significantly augmented the in vivo antitumoral efficacy of synthetic T cells in the KP tumor-bearing mice. FIG. 9D shows the survival curves of the tumor-bearing mice cohorts treated with the indicated regimens. FIG. 9E shows a graph monitoring the progression of tumor growth in CT26 tumor-bearing mice treated as indicated, measured via IVIS. Animal cohorts were treated with: PBS (intravenous injection); GzmB; nFLIP (non-cleavable FLIP, intravenous injection); FLIP (intravenous injection); CFLIP (MMP-activated conditional FLIP, intravenous injection); PD-1 (anti-PD1 immune checkpoint inhibitor, intraperitoneal injection); PD-1/nFLIP (anti-PD1 immune checkpoint inhibitor, in combination with a non-cleavable FLIP, intravenous injection); PD-1/FLIP (anti-PD1 immune checkpoint inhibitor, intraperitoneal injection, in combination with a FLIP, intravenous injection); PD-1/CFLIP (anti-PD1 immune checkpoint inhibitor, intraperitoneal injection, in combination with an MMP-activated conditional FLIP, intravenous injection). Significant antitumor efficacy was observed with both synthetic T cell monotherapy and the combination of synthetic T cell therapy with anti-PD1 immune checkpoint inhibitors. FIGS. 9F and 9G show survival curves of CT26 tumor-bearing mice cohorts treated with the indicated regimens.

FIGS. 10A and 10B demonstrate the ability to target Cy-7-tagged 8-arm polyethylene glycol nanoparticles to specific organs in vivo. FIG. 10A shows a graph of the accumulation of Cy-7-tagged 8-arm polyethylene glycol nanoparticles in the kidney when engineering the protease substrate to be cleaved by an enzyme expressed only in the kidney (kidney cleavable peptide, KCP). The Cy7-tagged nanoparticles upon cleavage by kidney-specific proteases increased interactions with renal cells and/or tissues. FIG. 10B shows organs imaged from mice treated with (right) or without (left) KCP-specific nanoparticles. Benchtop LI-COR IVIS was used to image fluorescent dye Cy7 that labeled nanoparticles.

FIG. 11 shows confocal microscopy images demonstrating that MMP-activatable CFLIPs regain fusogenicity upon activation via MMP9. The conjugation of fusion inhibitor 14 (see Table 2) to FLIP20 (to form CFLIP20) significantly suppresses the interaction between mouse KP lung cancer cells and liposomes. Upon in vitro activation by MMP (i.e., fusion inhibitors were cleaved off), the liposomes (termed pre-activated CFLIP20) regained fusogenic properties as indicated. Nuclei were stained with Hoechst 33342; lysosomes were stained with LysoTracker Green DND-26; cellular plasma membrane was stained with CellBrite Steady 650 membrane staining kit; CFLIPs were labeled with lipophilic Dil.

FIGS. 12A-12C demonstrate the contribution of particle size to fusion capacity of FLIPs. FIG. 12A depicts representative confocal images of colocalization of FLIPs of different particle sizes with KP mouse lung cancer cells. Nuclei (white arrows) were stained with Hoechst 33342; lysosomes were stained with LysoTracker Green DND-26 (open arrows); CFLIPs were labeled with lipophilic Dil (dashed arrows). FIG. 12B shows the Pearson correlation coefficient (PCC) of FLIPs of different particle sizes with endolysosomes as quantified from confocal images. FIG. 12C shows the normalized mean fluorescence intensity (FL) per cell of FLIPs with different sizes (normalized to the FL per cell of 76 nm FLIPs. Statistical analysis was performed using one-way ANOVA with Tukey's multiple comparison to the 76 μm FLIP. *p<0.05, **p<0.01, ***p<0.001, and ****p<0001.

FIGS. 13A-13B demonstrate the use of various types of molecules as inhibitors of FLIP membrane fusion. Fusion inhibitors or promoters were conjugated to FLIPs via a MMP peptide substrate(S). Fusion modulators used were: peptides (polyglutamic acid [e8], D-isomer amino acid, iRGD, poly(glutamic acid-co-lysine) [(ek)₅]), polyethylene glycol 2000 Da (PEG2k), and single stranded DNA 10mer (DNA10). FIG. 13A shows fluorescent confocal microscopy images of cells incubated with CFLIPs containing the various fusion modulators. Nuclei (white arrows) were stained with Hoechst 33342; lysosomes were stained with LysoTracker Green DND-26 (open arrows); CFLIPs were labeled with lipophilic Dil (dashed arrows). FIG. 13B shows the quantification of fusion efficiency of the indicated CFLIPs normalized to the fusion efficiency of an unmodified FLIP with the same liposome composition, indicating a significant decrease in fusogenicity with the addition of es and (ek) 5 peptides, as well as PEG2k and DNA10. Statistical analysis was performed using one-way ANOVA with Dunnett's multiple comparisons. *p<0.05. **p<0.01, ***p<0.001, and ****p<0.0001.

FIGS. 14A-14B demonstrate that endocytosis inhibitors are unable to inhibit cytosolic delivery of proteins via CFLIPs. FIG. 14A depicts representative confocal microscopy images showing the colocalization of endolysosomes with Cy5-labeled bovine serum albumin (BSA) (dashed arrows) delivered to cells with pre-activated CFLIPs (top row) or non-fusogenic liposomes (bottom row) in the presence of various inhibitors of endocytosis. Nuclei (white arrows) were stained with Hoechst 33342, and lysosomes (black arrows) were stained with Lyso Tracker Green DND-26. Endocytosis inhibitors used were: amilorate (AMI), chlorpromazine (CPZ), and filipin complex III (FIL). FIG. 14B shows the quantification of Mander's overlap coefficient (MI) of delivered Cy5-tagged BSA over endosomal trackers. Statistical analysis was performed using unpaired Student's t-test. *p<0.05, **p<0.01, ***p<0.001, and ****p<0.0001.

FIG. 15 shows hematoxylin and eosin (H&E) staining of KP lung transplant tumors treated using different delivery methods. Tumor-bearing mice were treated with PBS, granzyme B (GzmB), non-cleavable fusogenic liposomes with GzmB encapsulated (N-FLIP/GzmB), fusogenic liposomes with GzmB (FLIP/GzmB), MMP9-cleavable CFLIP/GzmB, an antibody against programmed cell death protein 1 (αPD-1), or a combination of CFLIP/GzmB and αPD-1 for 5 doses at 3 mg/kg of GzmB and 10 mg/kg of aPD-1. The lungs were harvested at a terminal endpoint according to euthanasia criteria or 2 days past the final treatment, whichever came first. Scale bar=5 mm.

FIGS. 16A-16G show that cryoprotectants allow for maintenance of reconstituted CFLIP formulations after freezing and lyophilization. FIG. 16A shows the size of CFLIPs measured before (Original) and after freezing (Frozen) and lyophilization (Lyophilized). Original means untreated CFLIPs (in 1×PBS; or say before treatment), frozen indicates CFLIP reconstituted from frozen CLFIPs (simply thaw frozen samples), and lyophilized indicates reconstituted lyophilized CFLIPs in deionized water (i.e., dissolve lyophilized powders in DI water). Sucrose was added as the sugar excipient at the indicated w/v percentages. The addition of 25% sucrose (25% w/v) allowed for nearly perfect preservation of CFLIP size, polydispersity, and zeta potential following freezing or lyophilization, FIG. 16B demonstrates example cryo-transmission electron micrographs of original CFLIP2 and its counterparts reconstituted from frozen and lyophilized formulations. Scale bar=100 nm. Quantifications of size, polydispersity index, and zeta potential before and after freezing and lyophilization are shown when using 25% trehalose (FIG. 16C) 25% lactose (FIG. 16D), or 18% D-mannitol (FIG. 16E) as the cryoprotectant. Compared to trehalose and mannitol, which failed to retain the stability of the CFLIPs following freezing and lyophilization, lactose enabled high levels of preservation of the CFLIPs during these processes. FIG. 16F depicts dynamic light scattering of CFLIP2 (original=reconstituted from frozen and lyophilized formulations) in the presence of 18% D-mannitol. FIG. 16G shows the quantification of leakage of bovine serum albumin (BSA, used in this experiment as a model protein) from CFLIP2 after freezing and lyophilization in the presence of 25% sucrose as a cryoprotectant.

### DETAILED DESCRIPTION OF THE INVENTION

It is currently challenging to deliver semi- or impermeable therapeutics intracellularly for efficacious responses. Direct systemic administration naked biomolecules and synthetic molecules are often limited by enzymatic degradation, insufficient bioavailability at the disease site and off-target side effects in healthy tissues. Moreover, these biomolecules are often impermeable to cell membranes. Even when the cargos are successfully transported into cells, only about 2% of the drugs are capable of escaping early endosomes to reach intended intracellular targets. Without being bound by a particular theory, the reagents disclosed herein may increase the disease-site targeting efficiency of cargo and precise delivery of biomolecules to subcellular compartments of action, which may be useful for the development of fast emerging precision medicines and accelerate clinical translation for patients with life threatening conditions.

Protease dysregulation is an important hallmark of both infectious diseases and non-communicable diseases.^{3,4} Upregulation of certain proteases is broadly observed in events such as angiogenesis, tumor invasion, metastasis, inflammation, and thrombosis.^{5,6} Aspects of the present disclosure provide reagents, including conditional fusogenic liposomal platforms (CFLIPs), that deliver therapeutics including proteins and peptides intracellularly in response to aberrant proteases at disease sites. In some embodiments, a conditional fusogenic liposomal platform comprises a liposome with a cationic lipid and the liposome is linked to a fusion inhibitor. In some embodiments, CFLIPs are a nanoparticle system that comprises of therapeutic encapsulating fusogenic liposomes decorated with fusion inhibitors via protease sensitive substrates.

The reagents disclosed herein, in some aspects, provide a conditionally activatable delivery system that i) responds to disease hallmarks, and ii) allows for targeted cytosolic transport of vulnerable therapeutics with limited permeability into the cells. Upon selection activation by dysregulated endogenous triggers in the pathological sites, the reagents disclosed herein may deliver the encapsulated therapeutics directly to cytosols not through endocytoses (major pathways in which nanomedicines are internalized by cells), but via fusion with plasma membranes. Non-limiting examples of drugs include antibodies, proteolysis targeting chimera (PROTAC), enzymes, peptides, nucleic acids, intrabodies, transcription factors, ribonucleoprotein particles (RNPs) and small molecules (e.g., trabectedin), and the hallmarks of the disease include but not limited to aberrant proteases, pH values, redox and hypoxia. Activatable fusogenicity, in some embodiments, addresses many of the limitations of conventional targeted nanoparticle delivery systems, including the challenges for delivering vulnerable and cell-impermeable large biomolecules.

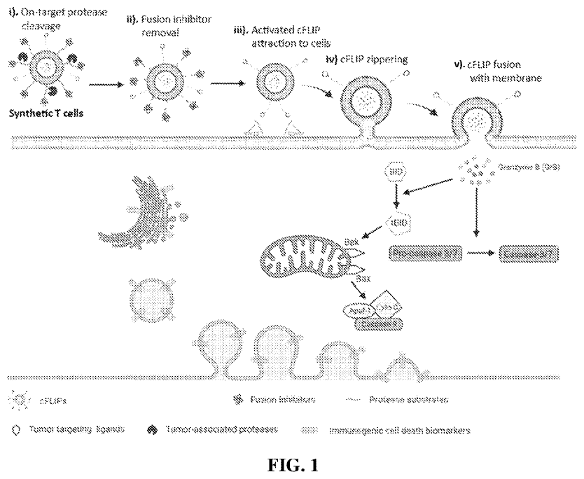

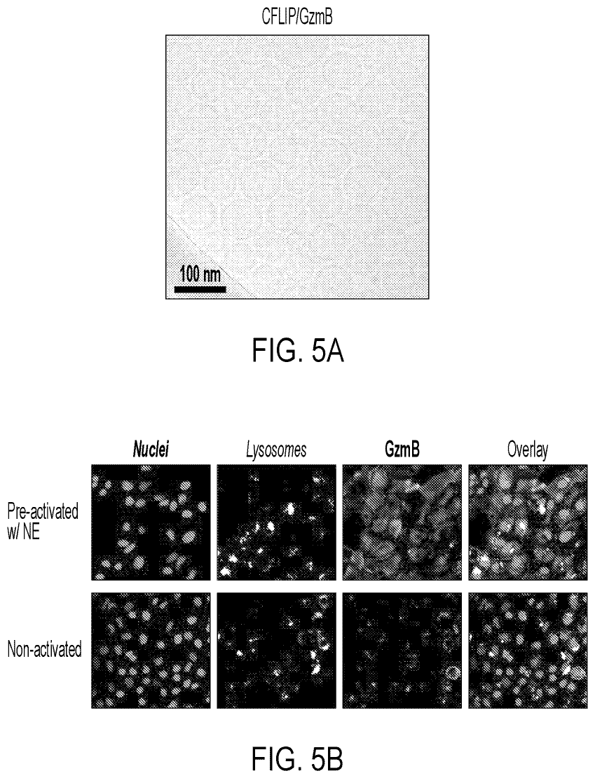

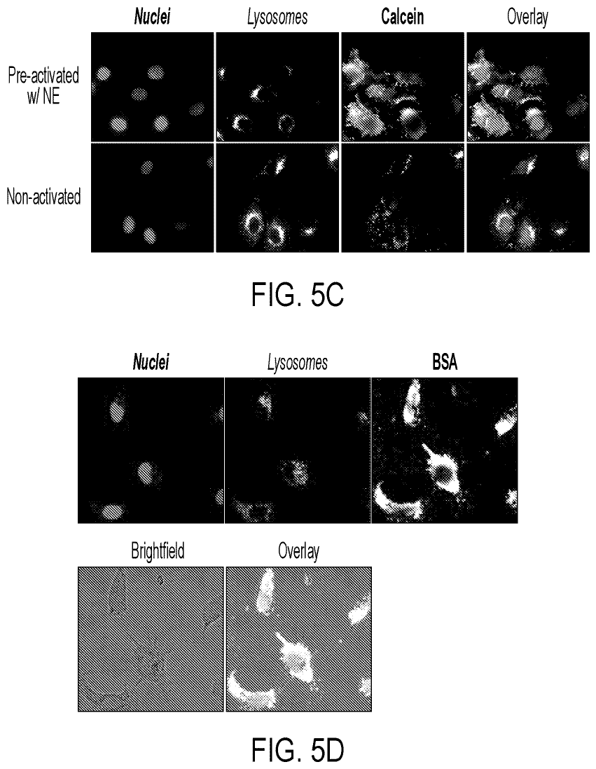

The Examples described herein show that CFLIPs exhibit reduced interaction with cells but in the event of internalization, the encapsulated cargo remained largely endocytosed in the liposome in the absence of protease activation. The fusogenic properties of the CFLIPS were switched on in the presence of upregulated proteases at disease sites in vivo-bypassing endocytic entrapping in the cells. This endocytosis-independent cellular uptake indicates that the CFLIPs may be utilized for targeted cytosolic delivery of those drugs vulnerable to degradation or entrapment in endosomes and lysosomes. Therefore, in some embodiments, cargo including molecules with limited permeability, including peptide or protein drugs, may be incorporated in the CFLIPs formulated with specific protease-cleavable substrates and fusion inhibitors. As a non-limiting example, a reagent comprising a liposome with a cationic lipid and a lipid conjugated to a fusion inhibitor via a protease substrate delivered granzyme B into the cytosols of cells, mimicking the function of cytotoxic T lymphocytes and natural killer cells in programmed cell death (FIGS. 1 and 5). This delivery of granzyme B occurred in a protease-activatable and pore-forming motif-free (e.g. perforin, gasdermin) manner. The reagents disclosed herein escorted the granzyme B into the cytosol, without the need for pore-forming proteins natively employed by endogenous cytotoxic T lymphocytes. In some embodiments, the reagents of the disclosure could function as synthetic T cells. In some embodiments, the described reagents overcome challenges in delivery efficiency by bypassing endocytic pathways. By encapsulating granzyme B, the conditional FLPs can potentially function as a ‘synthetic T cell’ for cancer immunotherapy applications. In comparison to endogenous T cell or NK cell therapy, the ‘synthetic T cell’ does not rely on perforin (not stable and calcium dependent) to mediate the transfer of granzyme B into the cytosol of the target cells but utilizes protease activities to function, which may improve delivery efficiency and reduce toxicity. Without being bound by a particular theory, the regents described herein, may, in some embodiments, be used to replace the need of perforin required for T cell functionality with a protease trigger.

Without being bound by a particular theory, through use of protease-responsive substrates, the Examples herein demonstrate not only that the amount of reagent accumulating at a disease site could be tuned but that the amount of reagent accumulating in tissues can be tuned, which may be useful in obtaining activation of fusion of the CFLIPs at target organs. In some embodiments, by using particular substrates that are cleaved by proteases upregulated at disease sites such as the tumor environment, the reagents disclosed herein may be activated to release therapeutic cargoes after the shedding of fusion inhibitor, while remain inactive at other organs. In some embodiments, the reagents of the present disclosure reduce the toxicity and side effects of the therapeutic of interests by encapsulating a biologically active agent or therapeutic and only releasing such cargo in the presence of an environmental trigger, including protease cleavage. For example, without being bound by a particular theory liposome-encapsulated proteins such as granzyme B will likely undergo endocytosis and subsequent degradation in lysosomes at the sites where protease triggers are absent or not upregulated, which may minimize its activity at non-target sites. In some embodiments, genomic editing proteins including CAS proteins may be encapsulated as cargo. In some embodiments, a reagent disclosed herein may be used for the protease triggered delivery of sgRNA and/or Cas 9, base editor (BE) at a target site.

Current approaches delivering protein therapeutics to intracellular targets include attachment of cell penetrating domains to cargos or delivery vehicle for direct translocation across plasma membranes,¹⁶ or use of nanoparticles to promote cargo internalization by target cells followed by endosomal escape. Alternatively, proteins can also be encoded in the form of messenger RNA encapsulated in non-viral or viral vehicle and are then produced intracellularly.¹⁴ Other methods of cytosolic drug delivery often require cell penetrating motifs, including perforins, cell penetrating peptides, or subcellular vesicle rupture. However, existing methods have several limitations that compromise delivery efficiency of these vehicle. For instance, many of the nanocarriers work better with particular cargo types (e.g., lipid nanoparticles for nucleic acids, but not for proteins or peptides). Moreover, these nanocarriers once in the cells must escape from endosomal compartments that trap nanocarriers and degrade loaded cargos enzymatically and the efficiency is limited by the subtle balance between inefficient endosomal rupture and toxicity caused by excessive endosomal disruption. Without being bound by a particular theory, in some embodiments, the liposomes linked to a fusion inhibitor disclosed herein have minimal interaction in their inactive form with surrounding cells (e.g. normal cells adjacent to disease sites and in distant tissues) that have a low abundance of protease expression. In some embodiments, in the absence of a protease a reagent disclosed herein will not fuse with the plasma membrane of a target cell and have minimal or insignificant interaction with cells. In some embodiments, an interaction with a cell is internalization by a cell. In some embodiments, in the absence of a protease, a reagent disclosed herein is not internalized in a cell.

In some embodiments, the reagents disclosed herein allow for the opportunity to co-encapsulate therapeutic cargoes of different types (e.g., nucleic acids, proteins, peptides, and/or small molecules, etc.). In some embodiments, the disclosed reagents and methods are facile and scalable in comparison to virus/cell-based approaches.

Cell penetrating peptides (CPPs) capable of crossing cell membrane have been widely harnessed as a carrier to mediate cytosolic delivery of a wide range of therapeutic cargos. For the vast majority of CPPs, however, cytosolic delivery efficiency remains poor. CPPs are often susceptible to proteolysis in vivo, and non-specific uptake at cellular and tissue levels, making them inefficient at delivering biomacromolecules across the cell membrane. In some embodiments, delivery of a therapeutic a protein is more desirable than delivery of a cargo a mRNA. The short lifetime of ribonucleoproteins (RNPs) in cells may be useful in limiting opportunities for off-target editing, as demonstrated by previous reports that delivering BE RNPs instead of BE-encoding DNA mRNA leads to substantially reduced off-target editing, typically without sacrificing on-target editing efficiency”.^{17,18,19} In some embodiments, the methods disclosed herein allow for direct cytosolic delivery of payloads via liposome-membrane fusion, which may open up new opportunities for drug delivery and biomedical applications. The payloads could include small molecules, peptides, intrabodies, proteins, protein-nucleic acid complexes, or combinations thereof.

In some embodiments, the disclosed reagents and methods allow for directly encapsulating protein-based drugs and the conditional release of the these drugs to reduce off-target toxicity unlike fusogenic porous silicon nanoparticles that have been used to encapsulate nucleic acids. See, e.g., Sailor et al. for description of fusogenic porous silicon nanoparticles encapsulating nucleic acids.²⁰

Accordingly, in some aspects the disclosure provides a composition comprising a reagent, wherein the reagent comprises a modular structure having a liposome linked to a fusion inhibitor. In some embodiments, a reagent comprises one or more features shown in Table 7. In some embodiments, a reagent comprises a sequence that is at least 70%, at least 80%, at least 90%, or is 100% identical to a sequence shown in Table 7. The comparison of sequences and determination of percent identity between two sequences can be accomplished using a mathematical algorithm. Techniques for determining identity are codified in publicly available computer programs. Exemplary computer software to determine homology between two sequences include, but are not limited to, the GCG program package (Devereux, J. et al. Nucleic Acids Research, 12 (1): 387, 1984), the BLAST suite (Altschul, S. F. et al. Nucleic Acids Res. 25:3389, 1997), and FASTA (Altschul, S. F. et al. J. Molec. Biol. 215:403, 1990). Other techniques include: the Smith-Waterman algorithm (Smith, T. F. et al. J. Mol. Biol. 147:195, 1981; the Needleman-Wunsch algorithm (Needleman, S. B. et al. J. Mol. Biol. 48:443, 1970; and the Fast Optimal Global Sequence Alignment Algorithm (FOGSAA) (Chakraborty, A. et al. Sci Rep. 3:1746, 2013).

A modular structure, as used herein, refers to a molecule having multiple domains. The modularity of the reagents disclosed herein allow for multiplexed applications and tailoring of the reagents for different uses. As a non-limiting example, it was found in the Examples described below that a few amino acid residues (e.g., 1-10 amino acids) that remain after protease cleavage did not have a measurable impact on fusogenic properties and drug delivery capabilities of the described platform. This enables the broad nomination of peptide substrates for disease and tissue targeting. Fusion inhibitors based on peptides and nucleic acids can be engineered to function as targeting ligands, reporters, barcodes and even as therapeutics for synergistic therapy or multifunctional application. In some embodiments, the fusion inhibitor is released in response to an environmental trigger, and the released fusion inhibitor may be used as a urinary reporter, allowing for theranostic applications and non-invasive cancer treatment monitoring.

### Fusion Inhibitors

As used herein, a fusion inhibitor is a moiety that prevents a liposome that is linked to the fusion inhibitor from fusing with another lipid bilayer. For example, a fusion inhibitor may prevent a liposome from fusing with a cell or another liposome. In some embodiments, a fusion inhibitor is a negatively charged short peptide, zwitterionic molecule, short oligonucleotide, polymeric chain, and/or protein.

In some embodiments, a fusion inhibitor comprises a peptide. In some embodiments, the peptide is 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 500, 600, 700, 800, 900, 1,000, 10,000, or more amino acids in length. In some embodiments, a fusion inhibitor comprises a peptide that is 5-25 (e.g., 5-10, 5-15, 5-20, 10-20, or 10-25) amino acids in length. In some embodiments, a fusion inhibitor comprises a peptide that is 8-17 amino acids in length.

In some embodiments, a fusion inhibitor comprises one or more types of amino acids. Amino acids may be classified by their hydrophobicity, polarity, charge, size, hydropathy, stereochemistry, and other attributes. See, e.g., Table 1. Hydrophobic amino acids include glycine, alanine, proline, valine, leucine, isoleucine, phenylalanine, methionine, tryptophan, tyrosine, and analogs thereof. Polar amino acids include serine, threonine, asparagine, glutamine, cysteine, and analogs thereof. Charged amino acids include positively charged amino acids and negatively charged amino acids. Positively charged amino acids include lysine, arginine, histidine, and analogs thereof. Negatively charged amino acids include aspartate, glutamate, and analogs thereof. Suitable amino acids include, without limitation, both the D- and L-isomers of the naturally-occurring amino acids, as well as non-naturally occurring amino acids. Without being bound by a particular theory, the overall charge of a fusion inhibitor may affect the fusion potential of a linked liposome. In some embodiments, a fusion inhibitor comprising one or more negatively charged amino acids reduces fusion by a liposome that is linked to the fusion inhibitor as compared to a hydrophobic peptide of the same length.

TABLE 1
Non-limiting examples of amino acids.

| | 3- | 1- | Side | Side-chain | |
|---|---|---|---|---|---|
| | Letter | Letter | Chain | charge | Hydropathy |
| Amino Acid | Code | Code | Polarity | (pH 7.4) | Index |
| Alanine | Ala | A | nonpolar | Neutral | 1.8 |
| Arginine | Arg | R | polar | Positive | −4.5 |
| Asparagine | Asn | N | polar | Neutral | −3.5 |
| Aspartic acid | Asp | D | polar | Negative | −3.5 |
| Cysteine | Cys | C | polar | Neutral | 2.5 |
| Glutamic acid | Glu | E | polar | Negative | −3.5 |
| Glutamine | Gln | Q | polar | Neutral | −3.5 |
| Glycine | Gly | G | nonpolar | Neutral | −0.4 |
| Histidine | His | H | polar | positive (10%); | −3.2 |
| | | | | neutral (90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyronsine | Tyr | Y | polar | neutral | −1.3 |
| Valine | Val | V | nonpolar | neutral | 4.2 |

In some embodiments, a peptide-based fusion inhibitor comprises less than 50% (e.g., less than 40%, less than 30%, less than 20%, less than 10%, less than 5%, or less than 1%) hydrophobic residues among amino acids of the peptide sequence. In some embodiments, a peptide-based fusion inhibitor comprises less than 25% hydrophobic residues among amino acids of the peptide sequence. In some embodiments, in a peptide-based fusion inhibitor that comprises more negatively charged amino acids, the percentage of hydrophobic residues in the fusion inhibitor is higher as compared to a peptide-based fusion inhibitor with less negatively charged amino acids. In some embodiments, a peptide fusion inhibitor comprises less than 50% hydrophobic amino acids in its sequence. Without being bound by a particular theory, a peptide with more than 50% hydrophobic amino acids or positively charged amino acids may not affect the fusion if not promoting it. In some embodiments, a liposome linked to the sequence GGPLGVRGKC (SEQ ID NO: 8) (comprising 30% hydrophobic amino acids and 20% basic amino acids) is capable of fusing with a target cell. In some embodiments, a reagent comprises fusion inhibitors linked to 7% of total lipids in the liposomes (which equals the % of DOPE-MAL).

In some embodiments, a fusion inhibitor has an overall net charge of negative 1, negative 2, negative 3, negative 4, negative 5, negative 6, negative 7, negative 8, negative 9, negative 10, negative 11, negative 12, negative 13, negative 14, negative 15, negative 16, negative 17, negative 18, negative 19, negative 20, negative 30, negative 40, negative 50, negative 60, negative 70, negative 80, negative 90, negative 100, negative 150, negative 200, negative 300, negative 400 negative 500, negative 600, negative 700, negative 800, negative 900, or negative 1000. In some embodiments, a fusion inhibitor has an overall net charge of negative 1 to negative 10. In some embodiments, a fusion inhibitor has an overall net charge of negative 1 to negative 20. In some embodiments, a fusion inhibitor has an overall net charge of negative 3 to negative 20. In some embodiments, a fusion inhibitor has an overall net charge of negative 4 to negative 20. In some embodiments, a fusion inhibitor has an overall net charge of negative 3 to negative 10. In some embodiments, a fusion inhibitor has an overall net charge of negative 4 to negative 10. In some embodiments, the overall net charge is determined at physiological pH. In some embodiments, physiological pH is 7.4.

In some embodiments, a fusion inhibitor comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 300, at least 400 at least 500, at least 600, at least 700, at least 800, at least 900, at least 1000 negatively charged amino acids. In some embodiments, a fusion inhibitor comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 30, at most 40, at most 50, at most 60, at most 70, at most 80, at most 90, at most 100, at most 150, at most 200, at most 300, at most 400 at most 500, at most 600, at most 700, at most 800, at most 900, at most 1000 negatively charged amino acids. In some embodiments, a fusion inhibitor comprises 1-10, 3-10, 4-10, 5-10, 6-10, 7-10, 8-10, or 9-10 negatively charged amino acids. In some embodiments, a fusion inhibitor comprises 1-20, 3-20, 4-20, 5-20, 6-20, 7-20, 8-20, 9-20, or 10-20 negatively charged amino acids. In some embodiments, the negatively charged amino acid is glutamate. In some embodiments, the negatively charged amino acid is aspartate.

In some embodiments, a fusion inhibitor comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 300, at least 400 at least 500, at least 600, at least 700, at least 800, at least 900, at least 1000 positively charged amino acids. In some embodiments, a fusion inhibitor comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 30, at most 40, at most 50, at most 60, at most 70, at most 80, at most 90, at most 100, at most 150, at most 200, at most 300, at most 400 at most 500, at most 600, at most 700, at most 800, at most 900, at most 1000 positively charged amino acids. In some embodiments, a fusion inhibitor comprises 1-10, 3-10, 4-10, 5-10, 6-10, 7-10, 8-10, or 9-10 positively charged amino acids. In some embodiments, a fusion inhibitor comprises 1-20, 3-20, 4-20, 5-20, 6-20, 7-20, 8-20, 9-20, or 10-20 positively charged amino acids. In some embodiments, the positively charged amino acid is lysine. In some embodiments, the positively charged amino acid is arginine. In some embodiments, the positively charged amino acid is histidine.

In some embodiments, a fusion inhibitor comprises a zwitterionic molecule. A zwitterionic molecule is a molecule that has a net formal charge of zero but comprises at least one component with a negative charge and at least one component with a positive charge. In some embodiments, a zwitterionic molecule is a peptide. In some embodiments, a zwitterionic molecule comprises a positively charged amino acid and a negatively charged amino acid. In some embodiments, a zwitterionic molecule comprises an even number of amino acids.

In some embodiments, a fusion inhibitor is a zwitterionic synthetic polymer, including but not limited to poly(carboxybetaine). In some embodiments, the zwitterionic synthetic polymer does not comprise PEG.

In some embodiments, a fusion inhibitor comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 300, at least 400 at least 500, at least 600, at least 700, at least 800, at least 900, at least 1000 D-isomer amino acids. In some embodiments, a fusion inhibitor comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 30, at most 40, at most 50, at most 60, at most 70, at most 80, at most 90, at most 100, at most 150, at most 200, at most 300, at most 400 at most 500, at most 600, at most 700, at most 800, at most 900, at most 1000 D-isomer amino acids. In some embodiments, a fusion inhibitor comprises 1-10, 3-10, 4-10, 5-10, 6-10, 7-10, 8-10, or 9-10 D-isomer amino acids. In some embodiments, a fusion inhibitor comprises 1-20, 3-20, 4-20, 5-20, 6-20, 7-20, 8-20, 9-20, or 10-20 D-isomer amino acids.

In some embodiments, a fusion inhibitor comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 300, at least 400 at least 500, at least 600, at least 700, at least 800, at least 900, at least 1000 L-isomer amino acids. In some embodiments, a fusion inhibitor comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 30, at most 40, at most 50, at most 60, at most 70, at most 80, at most 90, at most 100, at most 150, at most 200, at most 300, at most 400 at most 500, at most 600, at most 700, at most 800, at most 900, at most 1000 L-isomer amino acids. In some embodiments, a fusion inhibitor comprises 1-10, 3-10, 4-10, 5-10, 6-10, 7-10, 8-10, or 9-10 L-isomer amino acids. In some embodiments, a fusion inhibitor comprises 1-20, 3-20, 4-20, 5-20, 6-20, 7-20, 8-20, 9-20, or 10-20 L-isomer amino acids.

In some embodiments, a fusion inhibitor is a nucleic acid. In some embodiments, a fusion inhibitor is a single-stranded nucleic acid. In some embodiments, a fusion inhibitor is a single-stranded DNA or a single-stranded RNA molecule. In some embodiments, a fusion inhibitor is a double-stranded nucleic acid. In some embodiments, a fusion inhibitor is a double-stranded DNA or a double-stranded RNA molecule.

In some embodiments, a fusion inhibitor is a nucleic acid comprising one or more modified nucleotides, such as alternate or modified nucleobases. For example, for adenosine and guanosine nucleobases, alternate nucleobases can include bypoxanthine, xanthine, or 7-methylguanine, which correspond with the alternate nucleosides of inosine, xanthosine, and 7-methylguanosine, respectively. In addition, for example, cytosine, thymine, or uridine nucleobases, alternate nucleobases can include 5,6dihydrouracil, 5-methylcytosine, or 5-hydroxymethylcytosine, which correspond with the alternate nucleosides of dihydrouridine, 5-methylcytidine, and 5-hydroxymethylcytidine, respectively. Nucleobases may also include nucleobase analogues, for which a vast number are known in the art. Typically the analogue nucleobases confer, among other things, different base pairing and base stacking properties. Examples include universal bases, which can pair with all four canonical bases, and phosphate-sugar backbone analogues, such as peptide nucleic acids (PNAs), which affect the properties of the chain (PNAs can even form a triple helix). Nucleic acid analogues are also called “xeno nucleic acids” and represent one of the main pillars of xenobiology, the design of new-to-nature forms of life based on alternative biochemistries. Modified (e.g., artificial) nucleic acids include peptide nucleic acids (PNAs), morpholino and locked nucleic acids (LNAs), as well as glycol nucleic acid (GNA) and threose nucleic acid (TNA). Each of these is distinguished from naturally occurring DNA or RNA by changes to the backbone of the molecule. In some embodiments, a fusion inhibitor comprises a peptide nucleic acid. In some embodiments, a fusion inhibitor is a peptide nucleic acid.

In some embodiments, a fusion inhibitor is a modified nucleic acid comprising one or more intercalated bases.

In some embodiments, a fusion inhibitor is a modified nucleic acid comprising one or more nucleobase analogues. Example analogues include, but are not limited to inosine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenosine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxy methylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, 06-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms216A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (16A), 2-methylthio-N6-threonyl carbamoyladenine (ms216A), N6-methyl-N6-threonylcarbamoyladenine (m616A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

In some embodiments, a fusion inhibitor is a modified nucleic acid comprising one or more modified sugars. Example modified sugars include, but are not limited to 2′-deoxy fluoro (2FA), L-adenosine (LA), 2′-deoxyadenosine (dA), locked nucleic acid (LNA), 2′-methoxy (2Ome), 2′-methoxyethoxy (2MOE), 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, S′-biotin-ribose, 2′-O,4′-C-methylene-linked, 2′-O,4′-C-amino-linked ribose, and 2′-O,4′-C-thio-linked ribose. As referred to herein, L-adenosine (LA) refers to the enantiomer of D-adenosine. A locked nucleic acid is a nucleotide having a modified ribose moiety in which the ribose moiety comprises an extra bridge connecting the 2′ and 4′ carbons. This structure effectively “locks” the ribose in the 3′-endo structural conformation.

In some embodiments, a fusion inhibitor is a modified nucleic acid comprising one or more modified phosphate groups. Example modified phosphate groups include, but are not limited to phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

In some embodiments, the overall charge of the nucleic acid fusion inhibitor is negative.

In some embodiments, a nucleic acid fusion inhibitor comprises at least 5, at least 10, at least 15, at least 20, at least 25, at least 30, at least 35, at least 40, at least 45, at least 50, or more nucleotides. In some embodiments, a nucleic acid fusion inhibitor comprises 5-10, 10-15, 15-20, 20-25, 25-30, 30-35, 35-40, 40-45, or 45-50 nucleotides. In some embodiments, a nucleic acid fusion inhibitor comprises at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 22, at most 23, at most 24, at most 25, at most 26, at most 27, at most 28, at most 29, at most 30, at most 35, at most 40, at most 45, or at most 50 nucleotides. In some embodiments, a nucleic acid fusion inhibitor is 10 nucleotides in length.

In some embodiments, a fusion inhibitor comprises a fusion inhibitor set forth in Table 2. In some embodiments, a fusion inhibitor comprises a sequence that is at least 70%, at least 80%, at least 90%, or is 100%, including all values in between identical to a short synthetic peptide or zwitterionic molecule peptide sequence set forth in Table 2. In some embodiments, a fusion inhibitor comprises a short synthetic peptide or zwitterionic molecule peptide sequence set forth in Table 2. In some embodiments, a fusion inhibitor consists of a short synthetic peptide or zwitterionic molecule peptide sequence set forth in Table 2. In some embodiments, a fusion inhibitor comprises a sequence that is at least 70%, at least 80%, at least 90%, or is 100%, including all values in between identical to a nucleic acid sequence set forth in Table 2. In some embodiments, a fusion inhibitor comprises a nucleic acid sequence set forth in Table 2. In some embodiments, a fusion inhibitor consists of a nucleic acid sequence set forth in Table 2.

TABLE 2
Non-limiting examples of fusion inhibitors.

| | | Fusion inhibitor |
|---|---|---|
| Category of Fusion inhibitors | Code | (Peptide sequence indicated N-C) |
| Short synthetic peptides | I1 | eGVndneeGFFsAr (SEQ ID NO: 1) |
| | I2 | eeeeGVndneeGFFsAr (SEQ ID NO: 3) |
| | I3 | ececeece (SEQ ID NO: 4) |
| | I4 | eeeeeeeee (SEQ ID NO: 5) |
| Synthetic polymers | I5 | Polyethylene glycol 2000 |
| Zwitterionic molecules | I6 | kkeeekkeeekkeeck (SEQ ID NO: 6) |
| (peptides) | I7 | ekekekekek (SEQ ID NO: 7) |
| Single-stranded nucleic acids | I8 | T*C*A*T*A*G*T*T*A*G* (SEQ ID NO: 9) |

*denotes phosphorothioate (PS) backbone

In Table 2, a lower case letter in an amino acid sequences indicates a D-isomer amino acid.

In some embodiments, a fusion inhibitor comprises a peptide, protein, nucleic acid, and/or synthetic polymer. In some embodiments, a peptide is a synthetic peptide. In some embodiments, a fusion inhibitor comprises an antibody, single stranded nucleic acid, and/or a polymeric chain. In some embodiments, the nucleic acid is an oligonucleotide. In some embodiments, the nucleic acid comprises at least 2 nucleotides. In some embodiments, the antibody is a nanobody. In some embodiments, the nucleic acid comprises an overall negative charge. In some embodiments, a fusion inhibitor comprises a single-stranded oligonucleotide.

In some embodiments, a fusion inhibitor prevents a liposome that is linked to the fusion inhibitor from fusing with another lipid bilayer through steric hindrance. In some embodiments, a fusion inhibitor comprises polyethylene glycol (PEG). In some embodiments, a fusion inhibitor that prevents a liposome from fusing through steric hindrance is a molecules that is greater than 10 kDa in size. In some embodiments, a fusion inhibitor that prevents a liposome from fusing through steric hindrance is a molecules that is greater than 20 kDa in size. In some embodiments, a fusion inhibitor is 1000-4000 Da in molecular weight or larger. In some embodiments, a fusion inhibitor that is 1000-4000 Da in molecular weight has an overall net negative charge. In some embodiments, a fusion inhibitor that is 1000-4000 Da in molecular weight has an overall net neutral charge and is zwitterionic.

The term “antibody” encompasses whole antibodies (immunoglobulins having two heavy chains and two light chains), and antibody fragments. Antibody fragments include, but are not limited to, camelid antibodies, heavy chain fragments (VHH), Fab fragments, F(ab′) 2 fragments, nanobodies (single-domain antibodies), and diabodies (bispecific/bivalent dimeric antibody fragments). In some embodiments, the antibodies are monoclonal antibodies. Monoclonal antibodies are antibodies that are secreted by a single B cell lineage. In some embodiments, the antibodies are polyclonal antibodies. Polyclonal antibodies are antibodies that are secreted by different B cell lineages. In some embodiments, the antibodies are chimeric antibodies. Chimeric antibodies are antibodies made by fusing the antigen binding region (variable domains of the heavy and light chains, VH and VL) from one species (e.g., mouse) with the constant domain from another species (e.g., human). In some embodiments, the antibodies are humanized antibodies. Humanized antibodies are antibodies from non-human species whose protein sequences have been modified to increase their similarity to antibody variants produced naturally in humans. In some embodiments, the antibodies are fusion antibodies (e.g., fusion of VHH or other antibody fragments to other protein types). In some embodiments, the antibody is a humanized NANOBODY® onto which CDR grafting may be performed.

Non-limiting examples of monoclonal antibodies produced by the methods provided herein include abciximab (REOPRO®), adalimumab (HUMIRA®), alefacept (AMEVIVE®), alemtuzumab (CAMPATH®), basiliximab (SIMULECT®), belimumab (BENLYSTA®), bezlotoxumab (ZINPLAVA®), canakinumab (ILARIS®), certolizumab pegol (CIMZIA®), cetuximab (ERBITUX®), daclizumab (ZENAPAX, ZINBRYTA®), denosumab (PROLIA, XGEVA®), efalizumab (RAPTIVA®), golimumab (SIMPONI®), inflectra (REMICADE®), ipilimumab (YERVOY®), ixekizumab (TALTZ®), natalizumab (TYSABRI®), nivolumab (OPDIVO®), olaratumab (LARTRUVO®), omalizumab (XOLAIR®), ozoralizumab, palivizumab (SYNAGIS®), panitumumab (VECTIBIX®), pembrolizumab (KEYTRUDA®), rituximab (RITUXAN®), tocilizumab (ACTEMRA®), trastuzumab (HERCEPTIN®), secukinumab (COSENTYX®), and ustekinumab (STELARA®).

The reagent may include one or more types of fusion inhibitors. For instance each carrier may include 1 type of fusion inhibitor or it may include 2-1,000 different fusion inhibitors or any integer therebetween.

In some embodiments, the toxicity of cationic lipids is reduced through incorporation of a fusion inhibitor onto the liposome. In some embodiments, a fusion inhibitor increases the circulation time of a linked liposome, which may be useful in increasing tumor accumulation of the liposome and improve permeability and retention of encapsulated cargo.

In some embodiments, a fusion inhibitor may be used as a targeting moiety, reporter and/or synergistic therapeutic. In some embodiments, the fusion inhibitor is a therapeutic molecule. In some embodiments, a fusion inhibitor may be used as a barcode. For example, many treatment regimens do not offer the capabilities to also monitor disease progression. The fusion inhibitors disclosed herein may engineered to not only block the intracellular delivery but also function as synthetic biomarkers to be detected non-invasively. As shown in the examples below, one of the fusion inhibitors, Glu-1-Fibrinopeptide B, may be used for disease monitoring in urine samples. In some embodiments, a therapeutic agent may be used as a fusion inhibitor. For example, a fusion inhibitor may be engineered as therapeutic agents which upon liberation by an environmental trigger synergize with encapsulated drug cargos.

### Liposomes

Aspects of the present disclosure provide liposomes with a lipid linked to a fusion inhibitor. As used herein, liposomes are vesicles comprising one or more lipid bilayers. A lipid bilayer used herein comprises a lipid-based nanomaterial. In some embodiments, a lipid bilayer comprises a synthetic lipid-based nanomaterial. In some embodiments, a liposome is a small, spherical vesicle with a phospholipid bilayer that encapsulates an aqueous core.

Each bilayer of a liposome may encapsulate an aqueous compartment and may comprise two opposing monolayers of amphipathic lipid molecules. Amphipathic lipids may comprise a polar headgroup covalently linked to one or two non-polar acyl chains. Liposomes are often self-assembling structures. For example, energetically unfavorable contacts between the hydrophobic acyl chains and an aqueous environment often induce lipid molecules to rearrange; the polar headgroups orient towards the aqueous medium while the acyl chains orient towards the interior of the bilayer, which helps form a liposome. Liposomes may be made synthetically.

A liposome may have a single lipid bilayer (unilamellar liposomes, “ULVs”), or multiple lipid bilayers (multilamellar liposomes, “MLVs”), and can be made by any method known in the art. See, for example, U.S. Pat. Nos. 4,522,803, 4,588,578, 5,030,453, 5,169,637, 4,975,282, 4,235,871, and 5,008,050, the contents of which are incorporated herein by reference.

Various methods, including sonication, freeze-thaw, extrusion, microfluidic approaches, milling, and homogenization may be used to prepare liposomes, including using larger liposomes to make a liposome of a smaller size. See, e.g., U.S. Pat. No. 5,008,050. The size of a liposome may be determined by any known method, including but not limited to size exclusion chromatography and flow cytometry.

In some embodiments, a liposome comprises one or more cationic, helper, structural and/or functional lipids with phase transition lower or equal to room temperature. In some embodiments, room temperature is between 15 to 25° C. Non-limiting examples of lipids include 1,2-dioleoyl-3-trimethylammonium-propane (DOTAP), 1,2-dioleoyl-3-dimethylammonium propane (DODAP), 1,2-dimyristoyl-sn-glycero-3-phosphocholine (DMPC), 1,2-dimyristoyl-sn-glycero-3-phosphoethanolamine (DMPE), cholesterol, 1,2-dioleoyl-sn-glycero-3-phosphoethanolamine (DOPE), 1,2-dioleoyl-sn-glycero-3-phosphoethanolamine maleimide (DOPE-MAL), 1,2-distearoyl-sn-glycero-3-phosphoethanolamine maleimide (DSPE-MAL), 1,2-distearoyl-sn-glycero-3-phosphoethanolamine-N-[amino (polyethylene glycol)-2000] maleimide (DSPE-PEG2k-MAL), 1,2-distearoyl-sn-glycero-3-phosphoethanolamine-N-[amino(polyethylene glycol)-2000] DBCO (DSPE-PEG2k-DBCO), 1,2-dimyristoyl-rac-glycero-3-methoxypolyethylene glycol-2000 maleimide (DMG-PEG2k-MAL), DSPE-PEG2K, C14PEG2k, C14PEG1k, DMG-PEG1k, and DMG-PEG2k. See also, e.g., Table 3. In some embodiments, a lipid comprises polyethylene glycol 2000 Da (PEG2k). In some embodiments, a lipid is polyethylene glycol 2000 Da (PEG2k).

TABLE 3
Non-limiting examples of lipids for forming a liposome.

| Category | Lipid | Abbreviation |
|---|---|---|
| Cationic lipids | 1,2-dioleoyl-3-trimethylammonium-propane | DOTAP |
| | 1,2-dioleoyl-3-dimethylammonium propane | DODAP |
| Helper lipids | 1,2-dimyristoyl-sn-glycero-3-phosphocholine | DMPC |
| | 1,2-dimyristoyl-sn-glycero-3-phosphoethanolamine | DMPE |
| | Cholesterol | CHOL |
| Structural lipids | 1,2-dioleoyl-sn-glycero-3-phosphoethanolamine | DOPE |
| Functional lipids | 1,2-dioleoyl-sn-glycero-3-phosphoethanolamine | DOPE-MAL |
| | maleimide | |
| | 1,2-distearoyl-sn-glycero-3-phosphoethanolamine | DSPE-MAL |
| | maleimide | |
| | 1,2-distearoyl-sn-glycero-3-phosphoethanolamine-N- | DSPE-PEG2k-MAL |
| | [amino(polyethylene glycol)-2000] maleimide | |
| | 1,2-distearoyl-sn-glycero-3-phosphoethanolamine-N- | DSPE-PEG2k-DBCO |
| | [amino(polyethylene glycol)-2000] DBCO | |
| | 1,2-dimyristoyl-rac-glycero-3-methoxypolyethylene | DMG-PEG2k-MAL |
| | glycol-2000 maleimide | |

In some embodiments, a liposome comprises one or more cationic lipids. The term “cationic lipid” refers to lipids which carry a net positive charge at a pH less than 9, including but not limited to physiological pH (pH 7.2-7.4), tumor microenvironment pH (pH 6.6-pH 7.2), endosomal/lysosomal pH (pH 6.5-pH4.5), and gastrointestinal pH (pH 3-pH 1). Such lipids include, but are not limited to, DODAC, DOTMA, DDAB, DOTAP, DODAP, DC-Chol and DMRIE. Additionally, a number of commercial preparations of cationic lipids are available. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic lipids comprising DOGS in ethanol from Promega Corp., Madison, Wis., USA). A variety of methods are available for preparing liposomes e.g., U.S. Pat. Nos. 4,186,183, 4,217,344, 4,235,871, 4,261,975, 4,485,054, 4,501,728, 4,774,085, 4,837,028, 4,946,787; and PCT Publication No. WO 91/17424. The particles may also be composed in whole or in part of GRAS components. i.e., ingredients are those that are Generally Regarded As Safe (GRAS) by the US FDA. GRAS components useful as particle material include non-degradable food based particles such as cellulose.

Without being bound by a particular theory, lipid composition could be altered to affect liposomal physiochemical properties in terms of rigidity, surface charge, and reactivity. In some embodiments, in comparison to conventional liposomes and lipid nanoparticles, the reagents disclosed herein comprise a fusogenic liposomal platform (FLIPs) that directly fuses with the cell membrane (i.e., become part of the cell membrane), which may be useful in bypassing endocytic pathways. In some embodiments, a liposome comprises a lipid with low phase transition temperature. In some embodiments, a low phase transition temperature is a temperature below physiological temperature (e.g., a temperature below 99, below 98, or below 97 degrees Fahrenheit). Without being bound by a particular theory, membrane fluidity may promote fusion. In some embodiments, a membrane is kept fluid by incorporating phospholipids comprising an unsaturated fatty acid, cholesterol, proteins, and/or carbohydrates into the membrane. In some embodiments, a liposome may be moderately positive in the absence of a fusion inhibitor. In some embodiments, a liposome with a moderately positive charge can be used to promote electrostatic attraction to anionic cell membranes. In some embodiments, a moderate positive charge is +12 to +18 mV.

In some embodiments, liposomes are prepared with known extrusion method or microfluidic approaches. In some embodiments, fusion inhibitor and environmentally-responsive linker tandem construct is conjugated to phospholipids after the lipid film preparation. For example, conjugation of the fusion inhibitor and environmentally-responsive linker tandem construct may take place after liposome formation. In some embodiments, fusion inhibitor and environmentally-responsive linker tandem construct is conjugated to phospholipids before the lipid film preparation. For example, conjugation of the fusion inhibitor and environmentally-responsive linker tandem construct may take place before liposome formation and the purification follows liposome extrusion. In some embodiments, a fusion inhibitor and environmentally-responsive linker tandem construct comprises a fusion inhibitor linked to a protease substrate.

In some embodiments, a liposome comprises a particular ratio of lipids. In some embodiments, a liposome comprises DOTAP, DMPC, DOPE-MAL and/or DSPE-MAL. In some embodiments, a liposome comprises DOTAP, DMPC, DOPE-MAL and/or DSPE-PEG2K-MAL. In some embodiments, a liposome comprises DOTAP/DMPC/DOPE-MAL/DSPE-PEG2k-MAL at the following ratio: 20/70/7/3.

In some embodiments, liposome comprises more than 5% but less than 50% DOTAP. In some embodiments, liposome comprises more than 10% but less than 50% DOTAP. In some embodiments, liposome comprises more than 10% but at most 20% DOTAP. In some embodiments, liposome comprises less than 20% DOTAP. In some embodiments, a liposome comprises 10-20% DOTAP.

The liposome may serve as the core of the reagent. In some embodiments, a purpose of the liposome is to serve as a vesicle for carrying cargo. In some embodiments, the liposome is non-immunogenic, i.e. does not provoke an immune response in the body of the subject to which it will be administered. In some embodiments, a liposome may be linked to one or more ligands. For example, the liposome may be linked to a ligand to target the reagent to a tissue, cell or molecule.

In some embodiments, a liposome is less than 1.0 μm in diameter. A preparation of liposomes may include particles having an average liposome size of less than 1.0 μm in diameter. In some embodiments, a liposome is greater than 1.0 μm in diameter but less than 1 mm. A preparation of liposomes may include particles having an average liposome size of greater than 1.0 μm in diameter. The liposomes may therefore have a diameter of at least 5, at least 10, at least 25, at least 50, or at least 75 microns, including sizes in ranges of 5-10 microns, 5-15 microns, 5-20 microns, 5-30 microns, 5-40 microns, or 5-50 microns. A composition of liposomes may have heterogeneous size distributions ranging from 10 nm to mm sizes. In some embodiments the diameter is about 5 nm to about 500 nm. In other embodiments, the diameter is about 100 nm to about 200 nm. In other embodiment, the diameter is about 10 nm to about 100 nm. In some embodiments, a liposome is 50 to 100 nm in diameter. In some embodiments, a liposome is 70 to 80 nm in diameter. In some embodiments, a liposome is 5 to 100 nm in diameter. In some embodiments, a liposome is 100 to 200 nm in diameter. In some embodiments, a liposome is 100 to 150 nm in diameter. In some embodiments, a liposome is 150 to 200 nm in diameter. In some embodiments, a liposome is 76 nm in diameter. In some embodiments, a liposome is less than 150 nm (e.g., less than 140 nm, less than 130 nm, less than 120 nm, less than 110 nm, less than 100 nm, less than 90 nm, less than 80 nm, less than 70 nm, less than 60 nm, less than 50 nm, less than 40 nm, less than 30 nm, less than 20 nm, or less than 10 nm in diameter).

A plurality of liposomes may be homogeneous for one or more parameters or characteristics. A plurality that is homogeneous for a given parameter, in some instances, means that liposomes within the plurality deviate from each other no more than about +/−10%, preferably no more than about +/−5%, and most preferably no more than about +/−1% of a given quantitative measure of the parameter. In other instances, a plurality that is homogeneous means that all the liposomes in the plurality were treated or processed in the same manner, including for example exposure to the same agent regardless of whether every liposome ultimately has all the same properties. In still other embodiments, a plurality that is homogeneous means that at least 80%, preferably at least 90%, and more preferably at least 95% of liposomes are identical for a given parameter.

The plurality of liposomes may be heterogeneous for one or more parameters or characteristics. A plurality that is heterogeneous for a given parameter, in some instances, means that liposomes within the plurality deviate from the average by more than about +/−10%, including more than about +/−20%. Heterogeneous liposomes may differ with respect to a number of parameters including their size or diameter, their shape, their composition, their surface charge, their degradation profile, whether and what type of agent is comprised by the liposome, the location of such agent (e.g., on the surface or internally), the number of agents encapsulated by the liposome, etc. In some embodiments, there is separate synthesis of various types of liposomes which are then combined in any one of a number of pre-determined ratios prior to contact with the sample. As an example, in some embodiment, the particles may be homogeneous with respect to shape (e.g., at least 95% are spherical in shape) but may be heterogeneous with respect to size, degradation profile and/or agent comprised therein.

Particle size, shape and release kinetics can also be controlled by adjusting the liposome formation conditions. For example, particle formation conditions can be optimized to produce smaller or larger liposomes, or the overall incubation time or incubation temperature can be increased, resulting in liposomes which have prolonged release kinetics.

The liposomes may also be coated with one or more stabilizing substances, which may be particularly useful for long term depoting with parenteral administration or for oral delivery by allowing passage of the particles through the stomach or gut without dissolution. For example, liposomes intended for oral delivery may be stabilized with a coating of a substance such as mucin, a secretion containing mucopolysaccharides produced by the goblet cells of the intestine, the submaxillary glands, and other mucous glandular cells.

### Linkers

As used herein “linked” or “linkage” means two entities are bound to one another by any physicochemical means. Any linkage known to those of ordinary skill in the art, covalent or non-covalent, is embraced. Thus, in some embodiments the liposome has a linker (e.g., environmentally-responsive linker) attached to an external surface, which can be used to link the fusion inhibitor.

The reagents of the present disclosure comprise an environmentally-responsive linker that is located between the liposome and the fusion inhibitor. An environmentally-responsive linker, as used herein, is the portion of the reagent that changes in structure in response to an environmental trigger, e.g., in a subject, causing the release of a fusion inhibitor. Thus, an environmentally-responsive linker has two forms. The original form of the linker is attached to the liposome and the fusion inhibitor. When exposed to an environmental trigger the linker is modified in some way. For instance, it may be cleaved by an enzyme such that the fusion inhibitor is released. Alternatively, it may undergo a conformational change which leads to release of the fusion inhibitor.

In some embodiments, an environmentally-responsive linker is linked to a lipid on the liposome. In some embodiments, an environmentally-responsive linker is linker to a cationic lipid on the liposome. In some embodiments, an environmentally-responsive linker is linked to the surface of a liposome.

In some embodiments, an environmentally responsive linker is directly linking the fusion inhibitor to the liposome.

Certain environmental triggers present in a disease microenvironments have been associated with disease. For example, environmental triggers include enzymes, light, redox, hypoxia, pH, and temperature. In some embodiments, redox is a change in oxidation. An enzyme, as used herein refers to any of numerous proteins produced in living cells that accelerate or catalyze the metabolic processes of an organism. Enzymes act on substrates. The substrate binds to the enzyme at a location called the active site just before the reaction catalyzed by the enzyme takes place. Enzymes include but are not limited to proteases, glycosidases, lipases, heparinases, and phosphatases. In some embodiments, an environmentally-responsive linker is a protease substrate. In some instances, an environmentally-responsive linker comprises a photolabile group, which may change conformation in response to light (e.g., to a particular wavelength of light).

Dysregulated protease activities are implicated in a wide range of human diseases; including cancer, pulmonary embolism, inflammation, and infectious diseases, such as, bacterial infections, viral infections (e.g., HIV) and malaria. A reagent of the present disclosure may be used to detect an endogenous and/or an exogenous protease. An endogenous protease is a protease that is naturally produced by a subject (e.g., subject with a particular disease or a host with an infection). An exogenous protease is a protease that is not naturally produced by a subject and may be produced by a pathogen (e.g., a bacteria, a fungi, protozoa, or a virus). In some embodiments, a protease is only expressed by a subject (e.g., a human) and not by pathogen. In some embodiments, a protease is pathogen-specific and is only produced by a pathogen not by the pathogen's host.

Table 4 provides a non-limiting list of enzymes associated with (either increased or decreased with respect to normal) disease and in some instances, the specific substrate. In some embodiments, the enzyme-responsive linker is a tissue-specific substrate. In some embodiments, the enzyme-responsive linker is a disease-specific substrate. In some embodiments, the protease substrate is a organ-specific protease substrate. In some embodiments, the protease substrate is a tissue-specific protease substrate. In some embodiments, the protease substrate is a cell type-specific protease substrate. In some embodiments, the protease substrate is a disease-specific protease substrate. In some embodiments, the protease substrate is a platelet protease. In some embodiments, the cell, tissue, or organ is from eye, ear, nose, mouth, bone, lung, breast, pancreas, stomach, esophagus, muscle, liver, skin, heart, brain, kidney, testis, prostate, ovary, or intestine. In some embodiments, the cell, tissue, or organ is from blood. In some embodiments, an environmentally-responsive linker is a substrate of a protease that is overexpressed in the tumor microenvironment and/or by a tumor cell relative to a non-diseased cell. In some embodiments, a protease substrate is a MMP2 and/or MMP9 substrate, which may be used to detect several different cancers. In some embodiments, a protease substrate is an urokinase-type plasminogen activator.

Table 5 provides a non-limiting list of substrates associated with disease or other conditions. Numerous other enzyme/substrate combinations associated with specific diseases or conditions are known to the skilled artisan and are useful according to the invention.

TABLE 4
Non-limiting examples of disease-associated enzymes and substrates.

| Disease | Enzyme | Substrate |
|---|---|---|
| Cancer | MMP | collagens, gelatin, |
| | | various ECM proteins |
| Cancer | MMP-2 | type IV collagen and |
| | | gelatin |
| Cancer | MMP-9 | type IV and V |
| | | collagens and gelatin |
| Cancer | Kallikreins | kininogens, |
| | | plasminogen |
| Cancer | Cathepsins | broad spectrum of |
| | | substrates |
| Cancer | plasminogen activator, tPA | Plasminogen |
| Cancer | Urokinase-type plasminogen | Plasminogen |
| | activator, uPA | |
| Cancer | ADAM (A Diseintegrin And | various extracellular |
| | Metalloprotease, also MDC, | domains of |
| | Adamalysin) | transmembrane |
| | | proteins |
| Pancreatic carcinoma | MMP-7 | various, e.g. collagen |
| | | 18, FasL, HLE, DCN, |
| | | IGFBP-3, MAG, |
| | | plasminogen, other |
| | | MMPs |
| Pancreatic Cancer | ADAM9, ADAM15 | various extracellular |
| | | domains of |
| | | transmembrane |
| | | proteins |
| Prostate adenocarcinoma | Matriptase, a type II | unspecific, cleaves |
| | transmembrane serine protease | after Lys or Arg |
| | | residues |
| Prostate cancer | Kallikrein 3 | kininogens, |
| | | plasminogen |
| Prostate cancer | ADAM15 | various extracellular |
| | | domains of |
| | | transmembrane |
| | | proteins |
| Ovarian carcinoma | Kallikrein 6 | kininogens, |
| | | plasminogen |
| Epithelial-derived tumors | Matriptase, a type II | unspecific, cleaves |
| (breast, prostate, ovarian, colon, | transmembrane serine protease | after Lys or Arg |
| oral) | | residues |
| Ovarian Cancer | MMP-2, MMP-9, kallikrein-10 | type IV and V |
| | (hk-10) | collagens and gelatin, |
| | | kininogens, |
| | | plasminogen |
| Breast, gastric, prostate cancer | cathepsins B, L and D | broad spectrum of |
| | | substrates |
| Endometrial cancer | cathepsin B | unspecific cleavage of |
| | | a broad spectrum of |
| | | substrates without |
| | | clear sequence |
| | | specificity |
| esophageal adenocarcinoma | cathepsin B | unspecific cleavage of |
| | | a broad spectrum of |
| | | substrates without |
| | | clear sequence |
| | | specificity |
| Invasive cancers, metastases | type II integral serine proteases | |
| | (dipeptidyl peptidase IV | |
| | (DPP4/CD26), seprase/fibroblast | |
| | activation protein alpha | |
| | (FAPalpha) and related type II | |
| | transmembrane prolyl serine | |
| | peptidases)) | |
| Invasive cancers, metastases | Seprase | various ECM proteins |
| Viral Infections | | |
| All Retroviruses | viral protease | precursor GagPol |
| | | fusion |
| HIV | HIV protease (HIV PR, an | precursor Gag and |
| | aspartic protease) | GagPol proteins |
| Hepatitis C | NS3 serine protease | viral precursor |
| | | polyprotein |
| Dengue | Dengue protease | autocleavage |
| | | (NS2B/NS3), |
| | | NS3/NS4A and |
| | | NS4B/NS5 cleavage |
| West Nile | NS2B/NS3pro | viral precursor |
| | | polyprotein |
| Bacterial Infections | | |
| Legionella spp. | Zinc metalloprotease | MeO-Succ-Arg-Pro-Tyr |
| Meninogencephalitis | histolytic cysteine protease | |
| Streptococcus pyogenes (Group | streptococcal pyrogenic exotoxin | extracellular matrix, |
| A Streptococcus) | B (SpeB) | immunoglobulins, |
| | | complement |
| | | components |
| Clostridium difficile | Cwp84 | fibronectin, laminin, |
| | | vitronectin and other |
| | | ECM proteins |
| Pseudomonas aeruginosa | lasA | Leu-Gly-Gly-Gly-Ala |
| | | (SEQ ID NO: 11) |
| Pseudomonas aeruginosa | Large ExoProtease A | Cleavage of peptide |
| | | ligands on PAR1, |
| | | PAR2, PAR4 |
| | | (Protease-activated |
| | | receptor). See, e.g., |
| | | Kida et al, Cell |
| | | Microbiol. 2008 |
| | | July; 10(7): 1491-504. |
| Pseudomonas aeruginosa | protease IV | complement factors, |
| | | fibrinogen, |
| | | plasminogen (See, e.g., |
| | | Engel et al., J Biol |
| | | Chem. 1998 Jul. |
| | | 3; 273(27): 16792-7). |
| Pseudomonas aeruginosa | alkaline protease | Complement factor |
| | | C2 (See, e.g., Laarman |
| | | et al., J Immunol. 2012 |
| | | Jan. 1; 188(1): 386-93). |
| Additional Diseases | | |
| Alzheimer's disease | BACE-1,2 (Alzheimer secretase) | β-amyloid precursor |
| | | protein |
| Stroke and recovery | MMP, tPA | |
| cardiovascular disease | Angiotensin Converting Enzyme | angiotensin I, |
| | (ACE) | bradykinin |
| Atherosclerosis | cathepsin K, L, S | broad spectrum of |
| | | substrates |
| Arthritis | MMP-1 | triple-helical fibrillar |
| | | collagens |
| rheumatoid arthritis | thrombin | Osteopontin |
| Malaria | SUB1 | KITAQDDEES |
| | | (SEQ ID NO: 12) |
| osteoarthritis | thrombin | Osteopontin |
| osteoporosis/osteoarthritis | cathepsin K, S | broad spectrum of |
| | | substrates |
| Arthritis, inflammatory joint | Aggrecanase (ADAMTS4, | aggrecans |
| disease | ADAMTS11) | (proteoglycans) |
| thrombosis | factor Xa (thrombokinase) | Prothrombin |
| thrombosis | ADAMTS13 | von Willebrand factor |
| | | (vWF) |
| thrombosis | plasminogen activator, tPA | Plasminogen |
| Stress-induced Renal pressure | Prostasin | epithelial Na channel |
| natriuresis | | subunits |

TABLE 5
Non-limiting examples of substrates associated with disease and other conditions.

| DISEASE | TARGET SUBSTRATE | ENZYME |
|---|---|---|
| Inflammation | Interleukin 1 beta | MMP-2, MMP-3, MMP-9, |
| | | Trypsin, chymotrypsin, pepsin, |
| | | Lys-C, Glu-C, Asp-N, Arg-C |
| Pituitary gland | IGFBP-3 | MMP-1, MMP-3, MMP-9, |
| dysfunction, abnormal | | Trypsin, chymotrypsin, pepsin, |
| bone density, growth | | Lys-C, Glu-C, Asp-N, Arg-C |
| disorders | | |
| Cancer | TGF-beta | MMP-9, Trypsin, chymotrypsin, |
| | | pepsin, Lys-C, Glu-C, Asp-N, |
| | | Arg-C |
| Cancer, autoimmune | TNF | MMP-7, Trypsin, chymotrypsin, |
| disease | | pepsin, Lys-C, Glu-C, Asp-N, |
| | | Arg-C |
| Cancer, autoimmune | FASL | MMP-7, Trypsin, chymotrypsin, |
| disease | | pepsin, Lys-C, Glu-C, Asp-N, |
| | | Arg-C |
| Wound healing, cardiac | HB-EGF | MMP-3, Trypsin, chymotrypsin, |
| disease | | pepsin, Lys-C, Glu-C, Asp-N, |
| | | Arg-C |
| Pfeiffer syndrome | FGFR1 | MMP-2, Trypsin, chymotrypsin, |
| | | pepsin, Lys-C, Glu-C, Asp-N, |
| | | Arg-C |
| Cancer | Decorin | MMP-2, MMP-3, MMP-7, |
| | | Trypsin, chymotrypsin, pepsin, |
| | | Lys-C, Glu-C, Asp-N, Arg-C |
| Cancer | Tumor associated | Endoglycosidases |
| | carbohydrate antigens | |
| Cancer | Sialyl Lewis^{a} | O-glycanase |
| Cancer | Sialyl Lewis^{X} | O-glycanase |
| Cancer/ Rheumatoid | VEGF | Trypsin, chymotrypsin, pepsin, |
| Arthritis, pulmonary | | Lys-C, Glu-C, Asp-N, Arg-C |
| hypertension | | |
| Cancer | EGF | Trypsin, chymotrypsin, pepsin, |
| | | Lys-C, Glu-C, Asp-N, Arg-C |
| Cancer | IL2 | Trypsin, chymotrypsin, pepsin, |
| | | Lys-C, Glu-C, Asp-N, Arg-C |
| Cancer | IL6 | Trypsin, chymotrypsin, pepsin, |
| inflammation/angiogenesis | | Lys-C, Glu-C, Asp-N, Arg-C |
| Cancer | IFN-γ | Trypsin, chymotrypsin, pepsin, |
| | | Lys-C, Glu-C, Asp-N, Arg-C |
| Cancer | TNF-α | Trypsin, chymotrypsin, pepsin, |
| inflammation/angiogenesis, | | Lys-C, Glu-C, Asp-N, Arg-C |
| Rheumatoid Arthritis | | |
| Cancer, Pulmonary | TGF-β | Trypsin, chymotrypsin, pepsin, |
| fibrosis, Asthma | | Lys-C, Glu-C, Asp-N, Arg-C |
| Cancer, Pulmonary | PDGF | Trypsin, chymotrypsin, pepsin, |
| hypertension | | Lys-C, Glu-C, Asp-N, Arg-C |
| Cancer, pulmonary | Fibroblast growth factor | Trypsin, chymotrypsin, pepsin, |
| cystadenoma | (FGF) | Lys-C, Glu-C, Asp-N, Arg-C |
| Cancer | Brain-derived neurotrophic | Trypsin, chymotrypsin, pepsin, |
| | factor (BDNF) | Lys-C, Glu-C, Asp-N, Arg-C |
| Cancer | Interferon regulatory | Trypsin, chymotrypsin, pepsin, |
| | factors (IRF-1, IRF-2) | Lys-C, Glu-C, Asp-N, Arg-C |
| Inhibitor of tumor | MIF | Trypsin, chymotrypsin, pepsin, |
| suppressors | | Lys-C, Glu-C, Asp-N, Arg-C |
| Lymphomas/carcinomas, | GM-CSF | Trypsin, chymotrypsin, pepsin, |
| alveolar proteinosis | | Lys-C, Glu-C, Asp-N, Arg-C |
| Cancer invasion | M-CSF | Trypsin, chymotrypsin, pepsin, |
| | | Lys-C, Glu-C, Asp-N, Arg-C |
| Chemical carcinogenesis, | IL-12 | Trypsin, chymotrypsin, pepsin, |
| multiple sclerosis, | | Lys-C, Glu-C, Asp-N, Arg-C |
| rheumatoid arthritis, | | |
| Crohn's disease | | |
| Natural Killer T cell | IL-15 | Trypsin, chymotrypsin, pepsin, |
| leukemias, inflammatory | | Lys-C, Glu-C, Asp-N, Arg-C |
| bowel disease, rheumatoid | | |
| arthritis | | |
| Cirrhosis | Tissue inhibitor of MMPs | Trypsin, chymotrypsin, pepsin, |
| | (TIMPs) | Lys-C, Glu-C, Asp-N, Arg-C |
| Cirrhosis | Collagen I, III | MMP-1, MMP-8, Trypsin, |
| | | chymotrypsin, pepsin, Lys-C, |
| | | Glu-C, Asp-N, Arg-C |
| Cirrhosis | Collagen IV, V | MMP-2, Trypsin, chymotrypsin, |
| | | pepsin, Lys-C, Glu-C, Asp-N, |
| | | Arg-C |

In some embodiments, an environmentally-responsive linker comprises a protease substrate. In some embodiments, a protease substrate is an aspartic, glutamic, metallo-, cysteine, serine, and/or threonine protease substrate. In some embodiments, a protease substrate is a protease substrate shown in Table 6 or a protease substrate disclosed herein. In Table 6, lowercase letters denote D form amino acids, whereas uppercase letters denote L form amino acids.

TABLE 6
Non-limiting examples of protease substrates.

| Code | Protease-cleavable peptide sequence (N-C) |
|---|---|
| P1 | GGLVPRGSGC (SEQ ID NO: 13) |
| P2 | GGPVPLSLVMC (SEO ID NO: 14) |
| P3 | GGPLGVRGKC (SEQ ID NO: 8) |
| P4 | GGGSGRSANAKGC (SEQ ID NO: 15) |
| P5* | Nle(Obzl)-M(O)2-Oic-Abu-C |
| P6 | GGAAFAGC (SEQ ID NO: 16) |
| P7 | GGIPRSGGC (SEQ ID NO: 17) |
| K1 | GNSGRAVTYGC (SEQ ID NO: 18) |
| L1 | GKPISLISSGC (SEQ ID NO: 19) |
| L2 | GRPLALWESQGC (SEQ ID NO: 20) |
| G1 | GGAIEFDSGC (SEQ ID NO: 21) |
| S1 | GPMKRLTLGC (SEQ ID NO: 22) |
| P8** | CGGCRGDKGPDC (C2&C3 bridge) (SEQ ID NO: 23) |

*In P5, unnatural amino acids include 6-benzyloxynorleucine (Nle(O-Bzl)), methionine dioxide (Met(O)2), octahydroindolecarboxylic acid (OIC); and a-aminobutyric acid (Abu).
**In P8, second and third cysteines (C2 and C3, respectively ) form a bridge such that the sequence between C2 and C3 is a cyclic peptide.

Non-limiting examples of enzyme cleavable linkers may also be found in WO2010/101628, entitled METHODS AND PRODUCTS FOR IN VIVO ENZYME PROFILING, which was filed on Mar. 2, 2010.

In some embodiments, the environmentally-responsive linker is an enzyme-responsive linker, which is a portion of the modular structure that is connected to the carrier and can be modified by an enzyme.

In some embodiments, the enzyme-responsive linker is chosen depending on enzymes that are active in a specific disease state. For instance, tumors are associated with a specific set of enzymes. If the disease state being analyzed is a tumor then the product is designed with an enzyme-responsive linker that matches that of the enzyme expressed by the tumor or other diseased tissue. Alternatively, the enzyme specific site may be associated with enzymes that are ordinarily present but are absent in a particular disease state. In this example, a disease state would be associated with a lack or signal associated with the enzyme, or reduced levels of signal compared to a normal reference.

An enzyme, as used herein refers to any of numerous proteins produced in living cells that accelerate or catalyze the metabolic processes of an organism. Enzymes act on substrates. The substrate binds to the enzyme at a location called the active site just before the reaction catalyzed by the enzyme takes place. Enzymes include but are not limited to proteases, glycosidases, lipases, heparinases, phosphatases.

The enzyme-responsive linker may be optimized to provide both high catalytic activity (or other enzymatic activity) for specified target enzymes but to also release optimized detectable markers for detection. Patient outcome depends on the phenotype of individual diseases at the molecular level, and this is often reflected in expression of enzymes. The recent explosion of bioinformatics has facilitated exploration of complex patterns of gene expression in human tissues (Fodor, S. A. Massively parallel genomics. Science 277, 393-395 (1997)). Sophisticated computer algorithms have been recently developed capable of molecular diagnosis of tumors using the immense data sets generated by expression profiling (Khan J, Wei J S, Ringner M, Saal L H, Ladanyi M, Westermann F, et al. Classification and diagnostic prediction of cancers using gene expression profiling and artificial neural networks. Nat Med 2001; 7:673-679.). This information can be accessed in order to identify enzymes and substrates associated with specific diseases. Based on this information the skilled artisan can identify appropriate enzyme or substrates to incorporate into the reagent.

In some embodiments, the enzyme-responsive linker is a domain that is capable of being cleaved by a protease that is present (or upregulated) in a subject having a disease (e.g., cancer, metastatic cancer, an infection with a pathogenic agent, etc.). An enzyme-responsive linker may be incorporated into the liposome using well known teachings.

A disease microenvironment may have a pH that deviates from a physiological pH. Physiological pH may vary depending on the subject. For example, in humans, the physiological pH is generally between 7.3 and 7.4 (e.g., 7.3, 7.35, or 7.4). A disease microenvironment may have a pH that is higher (e.g., more basic) or lower (e.g., more acidic) than a physiological pH. As an example, acidosis is characterized by an acidic pH (e.g., pH of lower than 7.4, a pH of lower than 7.35, or a pH of lower than 7.3) and is caused by metabolic and respiratory disorders. Non-limiting examples of diseases associated with acidosis include cancer, diabetes, kidney failure, chronic obstructive pulmonary disease, pneumonia, asthma and heart failure. In some embodiments, an acidic pH induces cleavage of an environmentally-responsive linker and releases a fusion inhibitor from an reagent. Additional pH-responsive linkers include hydrazones and cis-Aconityl linkers. For example, hydrazones or cis-Aconityl linkers can be used to attach a fusion inhibitor (e.g., catalytic fusion inhibitor) to the liposome and the linker undergoes hydrolysis in an acidic environment.

Another non-limiting example of an environmentally-responsive linker is a temperature-responsive linker that changes structure at a particular temperature (e.g., a temperature above or below 37 degrees Celsius). In some instances, a temperature above 37 degrees Celsius (e.g., as indicative of a fever associated with influenza) induces cleavage of an environmentally-responsive linker and releases a fusion inhibitor from an reagent. In some embodiments, a temperature-responsive linker is linked (e.g., tethered) to a liposome.

In some embodiments, a temperature-responsive linker undergoes a conformational change in response to a particular temperature.

An environmentally-responsive linker (e.g., enzyme substrate, light-responsive, hypoxia-responsive, pH-responsive linker, or a temperature-responsive linker) may be attached directly to the liposome. For instance it may be coated directly on the surface of the liposome using known techniques. Alternatively if the liposome is a protein material it may be directly connected through a peptide bond. Additionally, the environmentally-responsive linker may be connected to the liposome through the use of another linker. Thus, in some embodiments the liposome may be attached directly to the environmentally-responsive linker or indirectly through another linker. The other linker may simply be a spacer (or in other works be a linker that is not responsive to an environmental trigger). Another molecule can also be attached to a linker. In some embodiments, two molecules are linked using a transpeptidase, for example Sortase A.

Examples of linking molecules include but are not limited to poly(ethylene glycol), peptide linkers, N-(2-Hydroxypropyl) methacrylamide linkers, elastin-like polymer linkers, and other polymeric linkages. Generally, a linking molecule is a polymer and may comprise between about 2 and 200 (e.g., any integer between 2 and 200, inclusive) molecules. In some embodiments, a linking molecule comprises one or more poly(ethylene glycol) (PEG) molecules. In some embodiments, a linking molecule comprises between 2 and 200 (e.g., any integer between 2 and 200, inclusive) PEG molecules. In some embodiments, a linking molecule comprises between 2 and 20 PEG molecules. In some embodiments, a linking molecule comprises between 5 and 15 PEG molecules. In some embodiments, a linking molecule comprises between 5 and 25 PEG molecules. In some embodiments, a linking molecule comprises between 10 and 40 PEG molecules. In some embodiments, a linking molecule comprises between 25 and 50 PEG molecules. In some embodiments, a linking molecule comprises between 100 and 200 PEG molecules.

In other embodiments, the second linker may be a second environmentally-responsive linker. The use of multiple environmentally-responsive linkers allows for a more complex interrogation of an environment and/or more precise delivery of cargo that is encapsulated in the liposome. For instance, a first linker may be sensitive to a first environmental condition or trigger and upon exposure to an appropriate trigger undergoes a conformational change which exposes the second environmentally-responsive linker. When a second trigger is also present then the second environmentally-responsive linker may be engaged in order to release the fusion inhibitor. In some embodiments, the fusion inhibitor is then be detected. Only the presence of the two triggers in one environment would enable the detection of the fusion inhibitor and/or deliver the encapsulated cargo to a target.

The sensitivity and specificity of an reagent may be improved by modulating presentation of the environmentally-responsive linker to its cognate environmental trigger, for example by varying the distance between the liposome and the environmentally responsive linker of the reagent. For example, in some embodiments, a polymer comprising one or more linking molecules is used to adjust the distance between a liposome and an environmentally-responsive linker, thereby improving presentation of the environmentally responsive linker to its cognate environmental trigger.

In some embodiments, the distance between a liposome and an environmentally-responsive linker (e.g., enzyme substrate, light-responsive linker, hypoxia-responsive linker, pH-responsive linker, or temperature-responsive linker) ranges from about 1.5 angstroms to about 1000 angstroms. In some embodiments, the distance between a liposome and an environmentally-responsive linker ranges from about 10 angstroms to about 500 angstroms (e.g., any integer between 10 and 500). In some embodiments, the distance between a liposome and a substrate ranges from about 50 angstroms to about 800 angstroms (e.g., any integer between 50 and 800). In some embodiments, the distance between a liposome and a substrate ranges from about 600 angstroms to about 1000 angstroms (e.g., any integer between 600 and 1000). In some embodiments, the distance between a liposome and a substrate is greater than 1000 angstroms.

In some embodiments, a reagent described herein comprises a spacer, which may be useful in reducing steric hindrance of an environmental trigger from accessing an environmentally-responsive linker. In some embodiments, a spacer comprises at least 1, 2, 3, 4, 5, 6, 7, 8, 9, 10, 11, 12, 13, 14, 15, 16, 17, 18, 19, 20, 30, 40, 50, 60, 70, 80, or 90 amino acids (e.g., glycine). In some embodiments, a spacer is a polyethelene glycol (PEG) spacer (e.g., a PEG spacer that is at least 100 Da, at least 200 Da, at least 300 Da, at least 400 Da, at least 500 Da, at least 600 Da, at least 700 Da, at least 800 Da, at least 900 Da, at least 1,000 Da, at least 2,000 Da, at least 3,000 Da, at least 4,000 Da, at least 5,000 Da, at least 6,000 Da, at least 7,000 Da, at least 8,000 Da, at least 9,0000 Da or at least 10,000 Da). In some embodiments, a PEG spacer is between 200 Da and 10,000 Da. In some embodiments, a spacer sequence is located between a liposome and an environmentally-responsive linker. In some embodiments, a spacer sequence is located between the environmentally-responsive linker and the fusion inhibitor.

Further, the size of the liposome may be adjusted based on the particular use of the reagent. For instance, the liposome may be designed to have a size greater than 5 nm. Liposomes, for instance, of greater than 5 nm are not capable of entering the urine, but rather, are cleared through the reticuloendothelial system (RES; liver, spleen, and lymph nodes). By being excluded from the removal through the kidneys any uncleaved reagent will not be detected in the urine during the analysis step. Additionally, larger liposomes can be useful for maintaining the liposome in the blood or in a tumor site where large liposomes are more easily shuttled through the vasculature. In some embodiments the liposome is 5 nm to 500 microns, 5 nm to 250 microns, 5 nm to 100 microns, 5 nm to 10 microns, 5 nm to 1 micron, 5 nm to 100 microns, 5 nm to 100 nm, 10 nm to 100 microns, 10 nm to 100 nm, 10 nm to 50 microns, 10 nm to 50 nm, or any integer size range therebetween. In other instances the liposome is smaller than 5 nm in size. In such instance the reagent will be cleared into the urine. However, the presence of free detectable marker can still be detected for instance using mass spectrometry. In some embodiments the liposome is 1-5 nm, 2-5 nm, 3-5 nm, or 4-5 nm.

Optionally the liposome may include a biological agent. In one embodiment a biological agent could be linked to the liposome or encapsulated by the liposome. In some embodiments, the liposome is linked to a detectable marker via an environmentally-responsive linker. In some embodiments, the detectable marker is a fusion inhibitor. Thus, the reagents of the present disclosure, in some embodiments, can achieve two purposes at the same time, the diagnostic methods and delivery of a therapeutic agent. In some embodiments the biological agent may be an enzyme inhibitor. In that instance, the biological agent can inhibit proteolytic activity at a local site and the detectable marker can be used to test the activity of that particular therapeutic at the site of action. HIV is an example of the disease in which active proteases can be monitored. In this embodiment the composition may include a micro-particle or other delivery device carrying a protease inhibitor. The protease susceptible site may be sensitive to the HIV proteases such that feedback can be provided regarding the activity of the particular protease inhibitor.

### Ligands

In some embodiments, a liposome is linked to a ligand capable of binding to a cell, which may improve the specificity and sensitivity of the reagents. In some embodiments, the ligand is linked to the surface of the liposome.

As used herein, a “ligand capable of binding to a cell”, or “capable of binding to a cell sample” refers to a molecule that specifically binds to a target cell. The ligand may be a peptide, protein (e.g., antibody), glycoprotein, binding protein, small molecule, nucleic acid (e.g., DNA, RNA, etc.), aptamer, etc. For example, in some embodiments a ligand is a peptide or protein that binds to a receptor on the surface of a particular cell type (e.g., a tumor cell). Examples of tissue targeting ligands include but are not limited to Lyp1, IRGD, anti-cancer antibodies (e.g., Trastuzumab, Pertuzumab, Brentuximab, Tositumomab, Ibritumomab, etc.) and fragments thereof, etc. in some embodiments, a ligand binds to a subset of cells in a particular tissue. In some embodiments, a ligand is capable of binding to a tissue.

In some embodiments, a ligand capable of binding to a cell includes one or more moieties that are capable of interacting with SNARE proteins on the membrane of a target cell. Such moieties may be useful in promoting attraction of a linked liposome to a target cell and also help promote fusion.

A “tumor-penetrating peptide” is a peptide that binds to a receptor expressed by a cancer cell and mediates internalization of a cargo molecule (e.g., a pro-diagnostic reagent) into the tumor tissue. In some embodiments, a tumor-penetrating peptide binds to a receptor involved in the active transport pathway of the cell (e.g., cancer cell), for example neuropilin 1 (NRP-1) or p32. Additional examples of receptors involved in the active transport pathway of cells (e.g., cancer cells) include but are not limited to neuropilin-2 (NRP-2), transferrin receptor, LDLR, etc.

Examples of tumor-penetrating peptides include but are not limited to LyP1 (CGNKRTRGC (SEQ ID NO: 24)), iRGD (CRGDKGPDC (SEQ ID NO: 2)), TT1, iNGR, and others for example as disclosed in Ruoslahti et al. J. Cell Biol. 188:759-768 (2010). In SEQ ID NO: 2, the two cysteines form a disulfide bridge. In some embodiments, a tumor-penetrating peptide comprises RGDKGPD (SEQ ID NO: 25). In some embodiments, one or more cysteine residues flank the tumor-penetrating peptide. In some embodiments, one or more spacer amino acids flank the tumor-penetrating peptide. In some embodiments, a suite of tumor-penetrating ligands specific for a range of primary receptors is produced by incorporation of the C-end rule motif, K/RXXK/R, which triggers the active internalization pathway of tumor cells.

### Compositions

Aspects of the present disclosure provide compositions comprising liposomes linked to a fusion inhibitor via an environmentally-responsive linker. In some embodiments, the liposome encapsulates cargo. In some embodiments, the cargo is a therapeutic molecule. In some embodiments, the cargo is a theranostic molecule. In some embodiments, the cargo is a detectable marker.

In some embodiments, the cargo is an antibody, a peptide, nucleic acids, a PI3K proteolysis targeting chimeric molecule (PROTAC), or a small molecule. In some embodiments, a PROTAC is a phosphorylated PROTAC. In some embodiments, a PROTAC comprises three domains: 1) a p85-binder, 2) a linker, and 3) an E3 ligase binder. In some embodiments, phosphorylation occurs at two labeled tyrosine residues (pY) on the peptide ligand, which enables the peptide, once in the cytosol, to bind to the p85 catalytic domain of PI3K without the activation of RTK. A linker is often used to connect the protein binder and E3 ligase binding domain in a PROTAC. In some embodiments, a linker is a polyethylene oxide (PEO) linker. In some embodiments, the PEO linker is (EO)ₓ, wherein x=2-6 and EO is ethylene oxide. In some embodiments, a linker is a C6 linker (CH₂)₆). In some embodiments, the cargo may further combination with a synergistic therapeutic. As a non-limiting example of a synergistic therapeutic application, granzyme B may be co-encapsulated in a liposome disclosed herein in combination with a reagent that inhibits a granzyme B inhibitor. In some embodiments, a granzyme B inhibitor is delivered using a reagent disclosed herein and a granzyme B inhibitor is co-delivered. In some embodiments, a reagent that inhibits a granzyme B inhibitor is an siRNA targeting a granzyme B inhibitor. In some embodiments, a reagent that inhibits a granzyme B inhibitor is an antisense oligonucleotide targeting a granzyme B inhibitor. In some embodiments, a granzyme B inhibitor is serpin B9.

In some embodiments, a cargo is a molecule that is greater than 1 kDa (e.g., greater than 2 kDa, greater than 3 kDa, greater than 4 kDa, greater than 5 kDa, or greater than 10 KDA, including any value in between) in size. In some embodiments, a therapeutic molecule is a therapeutic protein or a therapeutic nucleic acid. In some embodiments, a therapeutic nucleic acid encodes a protein. In some embodiments, a therapeutic nucleic acid is an antisense nucleic acid.

In some embodiments, a cargo is capable of inducing cell death in a target cell, which may help the reagent function as a synthetic T cell. In some embodiments, a cargo capable of inducing cell death in a target cell is granzyme B. In some embodiments, a cargo that is capable of inducing cell death is capable initiating apoptosis in a target cell.

In some embodiments, a composition is a pharmaceutical composition. Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. The agent may comprise different types of carriers depending on whether it is to be administered in solid, liquid or aerosol form, and whether it need to be sterile for such routes of administration as injection.

Aspects of the present disclosure provide compositions comprising any of the reagents disclosed herein in a solution comprising an excipient. In some embodiments, the excipient is a cryoprotectant. In some embodiments, the excipient is a sugar. In some embodiments, the excipient is sucrose. In some embodiments, the excipient is lactose. In some embodiments, the sugar is lactose. In some embodiments, the excipient is not trehalose. In some embodiments, the excipient is not D-mannitol.

In some embodiments, the solution comprising an excipient comprises 10-50% weight per volume (w/v) of a sugar. In some embodiments, the solution comprising an excipient comprises between 5% and 25% w/v of a sugar. In some embodiments, the solution comprising an excipient comprises about 25% w/v of a sugar. In some embodiments, the solution comprising an excipient comprises less than 50% w/v but more than 5% w/v of a sugar (e.g., between 5% and 45%, between 5% and 40%, between 5% and 35%, between 5% and 25%, between 10% and 45%, between 15% and 30%, between 20% and 30%, between 10% and 30%, or between 20% and 50% w/v of a sugar). In some embodiments, the sugar is sucrose. Without being bound by any particular theory, solutions comprising sucrose and/or lactose can be used as cryoprotectants to maintain the integrity of one or more characteristics of a reagent disclosed herein even when the reagent is lyophilized or frozen (e.g., at −80° C.). In some embodiments, use of sucrose and/or lactose as an excipient increases the stability of a reagent disclosed herein relative to when sucrose and/or lactose is not used as an excipient.

In some embodiments, a reagent disclosed herein that is frozen or lyophilized in a solution comprising sucrose and/or lactose maintains a diameter that is at least 70% but no more than 200% the diameter (e.g., maintains a diameter that is about 80%, about 90%, about 100%, 110%, about 120%, about 130%, about 140%, about 150%, about 160%, about 170%, about 180%, or about 190% the diameter) of the reagent prior to being frozen or lyophilized. In some embodiments, a reagent disclosed herein that is frozen or lyophilized in a solution comprising sucrose and/or lactose maintains a diameter that is between 80% and 200% (e.g., between 100% and 200%, between 90% and 200%, between 90% and 140%, between 100% and 150%, between 12% to 150%, including all values in between) the diameter of the reagent prior to being frozen or lyophilized.

In some embodiments, a reagent disclosed herein that is frozen or lyophilized in a solution comprising sucrose and/or lactose maintains a polydisperse index that is at least 70% but no more than 200% the polydisperse index (e.g., maintains a polydisperse index that is about 80%, about 90%, about 100%, 110%, about 120%, about 130%, about 140%, about 150%, about 160%, about 170%, about 180%, or about 190% the poly disperse index) of the reagent prior to being frozen or lyophilized. In some embodiments, a reagent disclosed herein that is frozen or lyophilized in a solution comprising sucrose and/or lactose maintains a polydisperse index that is between 80% and 200% (e.g., between 100% and 200%, between 90% and 200%, between 90% and 140%, between 100% and 150%, between 12% to 150%, including all values in between) the polydisperse index of the reagent prior to being frozen or lyophilized. In some embodiments, a reagent disclosed herein that is frozen or lyophilized in a solution comprising sucrose and/or lactose maintains a zeta potential that is at least 70% but no more than 200% the zeta potential (e.g., maintains a zeta potential that is about 80%, about 90%, about 100%, 110%, about 120%, about 130%, about 140%, about 150%, about 160%, about 170%, about 180%, or about 190% the zeta potential) of the reagent prior to being frozen or lyophilized. In some embodiments, a reagent disclosed herein that is frozen or lyophilized in a solution comprising sucrose and/or lactose maintains a zeta potential that is between 80% and 200% (e.g., between 100% and 200%, between 90% and 200%, between 90% and 140%, between 100% and 150%, between 12% to 150%, including all values in between) the zeta potential of the reagent prior to being frozen or lyophilized.

### Detectable Markers

In some embodiments, the reagent disclosed herein comprise a detectable marker. In some embodiments, the detectable marker is a fusion inhibitor. In some embodiments, a reagent comprises a separate detectable marker and a separate fusion inhibitor. In some embodiments, a detectable marker is capable of being released from the reagent when exposed to an enzyme in vivo. The detectable marker once released is free to travel to a remote site for detection. A remote site is used herein to refer to a site in the body that is distinct from the bodily tissue housing the enzyme where the enzymatic reaction occurs. In other words the remote site is a biological 1 sample or tissue that is different than the biological sample where the enzyme-responsive linker is administered and/or where the protease cleaves the molecule. In some embodiments, the bodily tissue housing the enzyme where the enzymatic reaction occurs is the blood or the tissue in a or surrounding a tumor. The remote site in some embodiments is urine.

In some embodiments, modification of the enzyme-responsive linker by an enzyme in vivo, results in the production of a detectable marker. The detectable marker may be composed of two ligands joined by a linker. The detectable marker may be comprised of, for instance one or more of a peptide, nucleic acid, small molecule, fluorophore/quencher, carbohydrate, particle, radiolabel, MRI-active compound, inorganic material, organic material, with encoded characteristics to facilitate optimal detection. The peptide itself may be the detectable maker, as it can be detected in the urine using known methods e.g. as described herein.

In some embodiments, an enzyme-responsive linker that comprises a capture ligand is a molecule that is capable of being captured by a binding partner. The detection ligand is a molecule that is capable of being detected by any of a variety of methods. While the capture ligand and the detection ligand will be distinct from one another in a particular detectable marker, the class of molecules that make us capture and detection ligands overlap significantly. For instance, many molecules are capable of being captured and detected. In some instances these molecules may be detected by being captured or capturing a probe. The capture and detection ligand each independently may be one or more of the following: a protein, a peptide, a polysaccharide, a nucleic acid, a fluorescent molecule, or a small molecule, for example. In some embodiments the detection ligand or the capture ligand may be, but is not limited to, one of the following: Alexa488, TAMRA, DNP, fluorescein, Oregon Green, Texas Red, Dansyl, BODIPY, Alexa405, Cascade Blue, Lucifer Yellow, Nitrotyrosine, HA-tag, FLAG-tag, His-tag, Myc-tag, V5-tag, S-tag, biotin or streptavidin. In some embodiments, the capture ligand and a detection ligand are connected by a linker. In some embodiments, the purpose of a linker between a capture ligand and a detection ligand is to prevent steric hinderance between the two ligands. Thus, the linker may be any type of molecule that achieves this. The linker may be, for instance, a polymer such as PEG, a protein, a peptide, a polysaccharide, a nucleic acid, or a small molecule. In some embodiments the linker is a protein of 10-100 amino acids in length. In other embodiments the linker is GluFib (SEQ ID NO. 1). Optionally, the linker may be 8 nm-100 nm, 6 nm-100 nm, 8 nm-80 nm, 10 nm-100 nm, 13 nm-100 nm, 15 nm-50 nm, or 10 nm-50 nm in length.

In some embodiments, the detectable marker is a ligand encoded reporter. Without wishing to be bound by any particular theory, a ligand encoded reporter binds to a target molecule, allowing for detection of the target molecule at a site remote from where the ligand encoded reporter bound to the target. In some embodiments, a ligand encoded reporter binds to a target molecule associated with a pathogenic agent. As used herein, “pathogenic agent” refers to a molecule that is indicative of the presence of a particular infectious agent (e.g., a virus, bacterium, parasite, etc.). Examples of pathogenic agents include viral proteins, bacterial proteins, biological toxins, and parasite-specific proteins (e.g., S. mansoni OVA protein).

In some embodiments, a detectable marker is a mass encoded reporter, for example an iCORE as described in WO2012/125808, filed Mar. 3, 2012, the entire contents of which are incorporated herein by reference. Upon arrival in the diseased microenvironment, the iCORE agents interface with aberrantly active proteases to direct the cleavage and release of surface-conjugated, mass-encoded peptide substrates into host urine for detection by mass spectrometry (MS) as synthetic biomarkers of disease.

The detectable marker may be detected by any known detection methods to achieve the capture/detection step. A variety of methods may be used, depending on the nature of the detectable marker. Detectable markers may be directly detected, following capture, through optical density, radioactive emissions, nonradiative energy transfers, or detectable markers may be indirectly detected with antibody conjugates, affinity columns, strepavidin-biotin conjugates, PCR analysis, DNA microarray, and fluorescence analysis.

The capture assay in some embodiments involves a detection step selected from the group consisting of an ELISA, including fluorescent, colorimetric, bioluminescent and chemiluminescent ELISAs, a paper test strip or LFA, bead-based fluorescent assay, and label-free detection, such as surface plasmon resonance (SPR). The capture assay may involve, for instance, binding of the capture ligand to an affinity agent.

The analysis step may be performed directly on the biological sample or the signature component may be purified to some degree first. For instance, a purification step may involve isolating the detectable marker from other components in the biological sample. Purification steps include methods such as affinity chromatography. As used herein an “isolated molecule” or “purified molecule” is a detectable marker that is isolated to some extent from its natural environment. The isolated or purified molecule need not be 100% pure or even substantially pure prior to analysis.

The methods for analysing detectable markers by identifying the presence of a detectable marker may be used to provide a qualitative assessment of the molecule (e.g., whether the detectable marker is present or absent) or a quantitative assessment (e.g., the amount of detectable marker present to indicate a comparative activity level of the enzymes. The quantitative value may be calculated by any means, such as, by determining the percent relative amount of each fraction present in the sample. Methods for making these types of calculations are known in the art.

The detectable marker may be labeled. For example, a label may be added directly to a nucleic acid when the isolated detectable marker is subjected to PCR. For instance, a PCR reaction performed using labeled primers or labeled nucleotides will produce a labeled product. Labeled nucleotides (e.g., fluorescein-labeled CTP) are commercially available. Methods for attaching labels to nucleic acids are well known to those of ordinary skill in the art and, in addition to the PCR method, include, for example, nick translation and end-labeling.

Labels suitable for use in the methods of the present invention include any type of label detectable by standard means, including spectroscopic, photochemical, biochemical, electrical, optical, or chemical methods. Preferred types of labels include fluorescent labels such as fluorescein. A fluorescent label is a compound comprising at least one fluorophore. Commercially available fluorescent labels include, for example, fluorescein phosphoramidides such as fluoreprime (Pharmacia, Piscataway, NJ), fluoredite (Millipore, Bedford, MA), FAM (ABI, Foster City, CA), rhodamine, polymethadine dye derivative, phosphores, Texas red, green fluorescent protein, CY3, and CY5, Polynucleotides can be labeled with one or more spectrally distinct fluorescent labels. “Spectrally distinct” fluorescent labels are labels which can be distinguished from one another based on one or more of their characteristic absorption spectra, emission spectra, fluorescent lifetimes, or the like. Spectrally distinct fluorescent labels have the advantage that they may be used in combination (“multiplexed”). Radionuclides such as 3H, 125I, 35S, 14C, or 32P are also useful labels according to the methods of the invention. A plurality of radioactively distinguishable radionuclides can be used. Such radionuclides can be distinguished, for example, based on the type of radiation (e.g. α, β, or δ radiation) emitted by the radionuclides. The 32P signal can be detected using a phosphoimager, which currently has a resolution of approximately 50 microns. Other known techniques, such as chemiluminescence or colormetric (enzymatic color reaction), can also be used.

Quencher compositions in which a “donor” fluorophore is joined to an “acceptor” chromophore by a short bridge that is the binding site for the enzyme may also be used. The signal of the donor fluorophore is quenched by the acceptor chromophore through a process believed to involve resonance energy transfer (RET). In some embodiments, cleavage of a peptide linker results in separation of the chromophore and fluorophore, removal of the quench, and generation of a subsequent signal measured from the donor fluorophore.

### Kits

Any of the reagents disclosed herein, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

### Methods of Use

The reagents and compositions described herein can be administered to any suitable cell, tissue, and/or organ. In some embodiments, a composition is administered to a subject. As used herein, a subject is a human, non-human primate, cow, horse, pig, sheep, goat, dog, cat, or rodent. In all embodiments human subjects are preferred. In aspects of the invention pertaining to cancer diagnosis and/or treatment in general the subject preferably is a human suspected of having cancer, or a human having been previously diagnosed as having cancer. Methods for identifying subjects suspected of having cancer may include physical examination, subject's family medical history, subject's medical history, biopsy, or a number of imaging technologies such as ultrasonography, computed tomography, magnetic resonance imaging, magnetic resonance spectroscopy, or positron emission tomography.

As used herein, a biological sample is a tissue sample. The biological sample may be examined in the body, for instance, by detecting a label at the site of the tissue, i.e. urine. Alternatively the biological sample may be collected from the subject and examined in vitro. Biological samples include but are not limited to urine, blood, saliva, or mucous secretion. In preferred embodiments the tissue sample is obtained non-invasively, such as the urine.

A “plurality” of elements, as used throughout the application refers to 2 or more of the elements.

In some embodiments, the reagents of the invention are administered to the subject in an effective amount for delivering cargo and/or detecting enzyme activity. An “effective amount”, for instance, is an amount necessary or sufficient to cause release of a detectable level of detectable marker in the presence of an enzyme. The effective amount of a compound of the invention described herein may vary depending upon the specific compound used, the mode of delivery of the compound, and whether it is used alone or in combination. The effective amount for any particular application can also vary depending on such factors as the disease being assessed or treated, the particular compound being administered, the size of the subject, or the severity of the disease or condition as well as the detection method. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective regimen can be planned.

In some embodiments, more than one reagent is administered to the subject. Some mixtures of reagents may include one or more fusion inhibitors, environmentally-responsive linkers, and/or cargo and other. Additionally a plurality of different reagents may be administered to the subject to target more than one cell, including more than one type of cell. In that instance, regents with different environmentally-responsive linkers, fusion inhibitors and/or types of cargo may be administered.

In some embodiments, the reagents and/or compositions are used to treat a subject. As used herein, the terms “treatment,” “treat,” and “treating” refer to reversing, alleviating, delaying the onset of, or inhibiting the progress of a disease or disorder, or one or more symptoms thereof, as described herein. In certain embodiments, treatment may be administered after one or more symptoms have developed. In other embodiments, treatment may be administered in the absence of symptoms. For example, treatment may be administered to a susceptible individual prior to the onset of symptoms or may be treated with another damaging agent (e.g., in light of a history of symptoms, in light of genetic or other susceptibility factors, a disease therapy, or any combination thereof). Treatment may also be continued after symptoms have resolved, for example, to prevent or delay their recurrence.

The reagents disclosed herein may be used in different disease contexts. In some embodiments, the reagents disclosed herein is administered to a subject with cancer. The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. In some embodiments, the subject has lung cancer. In some embodiments, the reagents disclosed herein may be useful as a cancer immunotherapy.

In some embodiments, the disease or condition treated and/or assessed according to the methods of the invention is any disease or condition that is associated with an enzyme. For instance, cancer, cardiovascular disease, arthritis, viral, bacterial, parasitic or fungal infection, Alzheimer's disease emphysema, thrombosis, hemophilia, stroke, organ dysfunction, any inflammatory condition, vascular disease, parenchymal disease, or a pharmacologically-induced state are all known to be associated with enzymes. A pharmacologically induced state is a condition in which enzyme inhibitors and other agents directly or indirectly affect enzyme activities. Thus each of the these can be assessed or monitored or studied according to methods of the invention.

In some embodiments, it is useful to be able to differentiate non-metastatic primary tumors from metastatic tumors, because metastasis is a major cause of treatment failure in cancer patients. If metastasis can be detected early, it can be treated aggressively in order to slow the progression of the disease. Metastasis is a complex process involving detachment of cells from a primary tumor, movement of the cells through the circulation, and eventual colonization of tumor cells at local or distant tissue sites. Additionally, it is desirable to be able to detect a predisposition for development of a particular cancer such that monitoring and early treatment may be initiated. For instance, an extensive cytogenetic analysis of hematologic malignancies such as lymphomas and leukemias have been described, see e.g., Solomon et al., Science 254, 1153-1160, 1991. Early detection or monitoring using the non-invasive methods of the invention may be useful.

Solid tumors progress from tumorigenesis through a metastatic stage and into a stage at which several different active proteases can be involved. Some protease are believed to alter the tumor such that it can progress to the next stage, i.e., by conferring proliferative advantages, the ability to develop drug resistance or enhanced angiogenesis, proteolysis, or metastatic capacity.

Accordingly, in some aspects, the disclosure provides a method for treating and/or detecting a tumor comprising administering to the subject having a tumor a reagent, wherein the reagent comprises a modular structure having a liposome linked to a fusion inhibitor via an enzyme-responsive linker, wherein the fusion inhibitor is a detectable marker and/or wherein the liposome is linked to a detectable marker via an enzyme-responsive linker, whereby the detectable marker is capable of being released from the reagent when exposed to a tumor-associated enzyme; obtaining a sample from the subject for detection of the detectable marker; and, analyzing the sample using a capture assay in order to detect the presence of the detectable marker, wherein the presence of the detectable marker in the sample is indicative of the subject having a tumor. In some embodiments, the liposome encapsulates a therapeutic molecule for treating the tumor. In some embodiments, the sample is a blood or urine sample.

In some embodiments, a reagent disclosed herein comprises a substrate for a pathogenic protease. In some embodiments, the liposome encapsulates a therapeutic molecule for treating an infection in which the pathogenic protease is expressed. In some embodiments, the sample is a blood or urine sample. Examples of infectious diseases that can be detected by methods and compositions of the disclosure include but are not limited to bacterial infections, viral infections, fungal infections, and parasitic infections.

In some embodiments, a reagent disclosed herein localizes to a cell, tissue, and/or organ more than another cell, tissue, and/or organ. In some embodiments, a reagent localizes to a cell, tissue, and/or organ at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1000% more as compared to another cell, tissue and/or organ. In some embodiments, the cell, tissue, and/or organ is in a subject.

In some embodiments, a method disclosed herein increases delivery of cargo to a cell, tissue, or organ expressing a protease relative to a cell, tissue, and/or organ with reduced expression of the protease. In some embodiments, a method disclosed herein increases delivery of cargo to a cell, tissue, or organ expressing a protease at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1000% more as compared to a cell, tissue, and/or organ with reduced expression of the protease.

In some embodiments, use of a reagent with a fusion inhibitor disclosed herein increases delivery of cargo to a cell, tissue, or organ relative to use of a reagent without the fusion inhibitor. In some embodiments, use of a reagent with a fusion inhibitor disclosed herein increases delivery of cargo to a cell, tissue, or organ at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1000% more as compared to use of a reagent without the fusion inhibitor.

In some embodiments, a reagent disclosed herein increases cell death of a target cell by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1000% as compared to a non-target cell. In some embodiments, the target cell is a disease cell.

In some embodiments, a method described herein includes assessment of a subject's response to a drug over a course of treatment. In some embodiments, a method comprises detecting a detectable marker that is released from the reagent in a sample from the subject. In some embodiments, the detectable marker is a fusion inhibitor.

Preferably the reagent and compositions thereof are injected into the body but could also be administered by other routes. For instance, the compounds of the present invention can be administered intravenously, intradermally, intraarterially, intralesionally, intratumorally, intracranially, intraarticularly, intraprostaticaly, intrapleurally, intratracheally, intranasally, intravitreally, intravaginally, intrarecially, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in creams, or by other method or any combination of the forgoing as would be known to one of ordinary skill in the art (see, for example, Remington's Pharmaceutical Sciences (1990), incorporated herein by reference).

### EXAMPLES

### Example 1: Synthesis of Conditional Fusogenic Liposomal Platforms (CFLIPs) and In Vitro Characterization of Protease Activation

FIG. 1 depicts a schematic showing conditional cytosolic delivery of cargo (in this schematic, Granzyme B) using synthetic T cells (i.e., CFLIPs) at a target cell membrane. The outer-facing lipids of the CFLIPs were conjugated with protease substrate-linked fusion inhibitors and targeting ligands. At target sites of interest, upregulated proteases quickly removed fusion inhibitors by cleaving the substrate linkers (step i). Shedding of the inhibitors (step ii) allowed the liposomes to fuse with cell membranes (steps iii and iv) for direct release of the encapsulated cargo (in these Examples, Granzyme B) into the cytosol (step v), bypassing endocytic pathways. In contrast, when in close proximity to a non-target cell membrane lacking aberrant proteolytic activity, the intact fusion inhibitors on the CFLIP surface dramatically suppressed fusogenicity of the CFLIPs. The CFLIPs were thus taken up by non-target cells predominantly via endocytosis but at a restricted rate, leading to quiescence of the cargo function. Overall, the CFLIPs enhanced on-target therapeutic responses with low off-target toxicity.

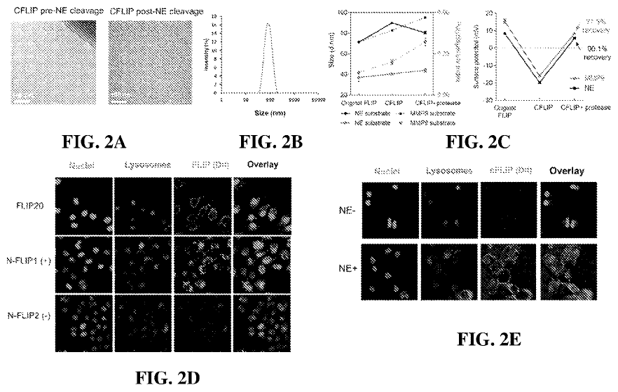

Since protease dysregulation is a hallmark of many non-communicable diseases such as cancer, these enzymatic activities were utilized as a smart trigger for drug release. The FLIPs were transformed to CFLIPs for activatable fusogenicity by covalently introducing fusion inhibitors removable through protease cleavage. Following protease activation, the CFLIPs achieved highly targeted cytosolic delivery of carried cargos (i.e., delivery to cytoplasm). A variety of approaches were used to obtain conditional activation of the CFLIPs, including charge conversion. Negatively charged peptides were used as a model inhibitor (e.g., I1-I4 in Table 2) to electrostatically veil cationic fusogenic lipids on the CFLIPs. Other fusion inhibitors included zwitterionic molecules (e.g., I6-I7 in Table 2) or synthetic polymers (e.g., I5 in Table 2). Functionalizing the liposomes with negatively charged peptides via substrate linkers to generate neutrophile elastase (NE)-activatable CFLIPs did not alter the geometry (FIG. 2A), hydrodynamic size (60-80 nm), or polydispersity (0.1-0.3) of the FLIPs (FIG. 2B). The surface charge of the CFLIPs turned moderately negative with the addition of a negatively charged inhibitory peptide and was nearly restored when protease triggers were added to remove the inhibitory peptides (FIG. 2C).

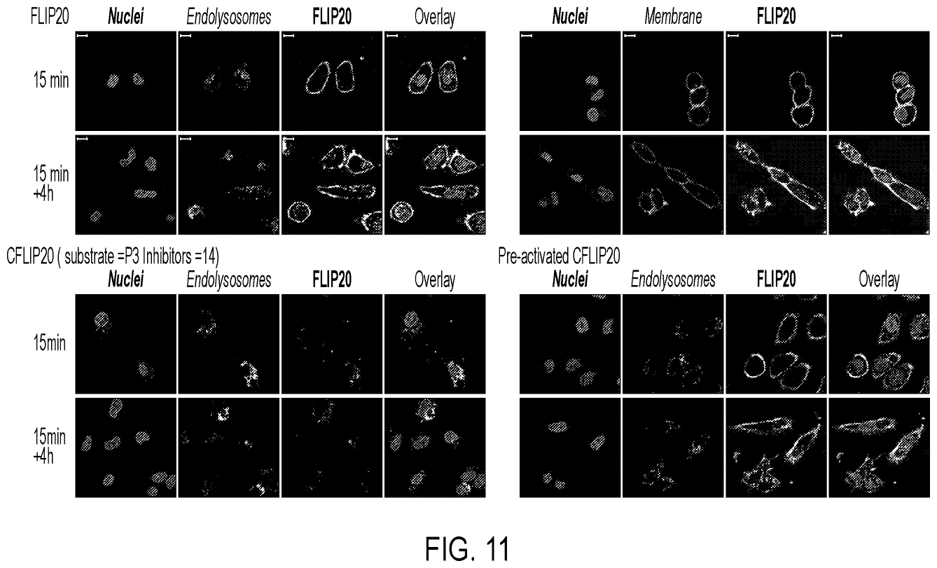

Negatively charged particles used as fusion inhibitors were very effective in manipulating the fusogenic characteristics of FLIPs. Confocal microscopy was performed using fluorescently-labeled FLIPs formulated as: FLIP20 (unmodified FLIP containing 20% DOTAP), N-FLIP1 (+) (non-fusogenic liposome with a positive surface charge), or N-FLIP2 (−) (non-fusogenic liposome with a negative surface charge). Fluorescent imaging revealed that non-fusogenic FLIPs (N-FLIP1 and N-FLIP2) were located mainly in the lysosomes or in the perinuclear area of cells, indicating suppressed electrostatic interactions with the cells (FIG. 2D, middle row and bottom row). Notably, functionalizing FLIP20 with negatively charged peptides via neutrophil elastase (NE)-sensitive substrate linkers to generate CFLIPs led to suppressed electrostatic interactions with the cells to levels comparable to non-fusogenic liposomes (i.e., N-FLIP1 and N-FLIP2) and completely blocked fusion with cell membranes (FIG. 2E, compare to FIG. 2D). In contrast, the CFLIPs pre-activated with NE to remove the negatively charged particles regained their fusogenic properties (FIG. 2E, bottom row). Cell membranes were labelled with a lipophilic dye (i.e., Dil) after incubation that was used to tag the lipid layers of CFLIPs. High levels of fusion were also observed when an MMP-responsive CFLIP was pre-activated with recombinant MMP9 (FIG. 11, pre-activated CFLIP20). The amino acid residues (2-4 amino acids) remaining on the CFLIP after cleavage did not restrict fusion activity.

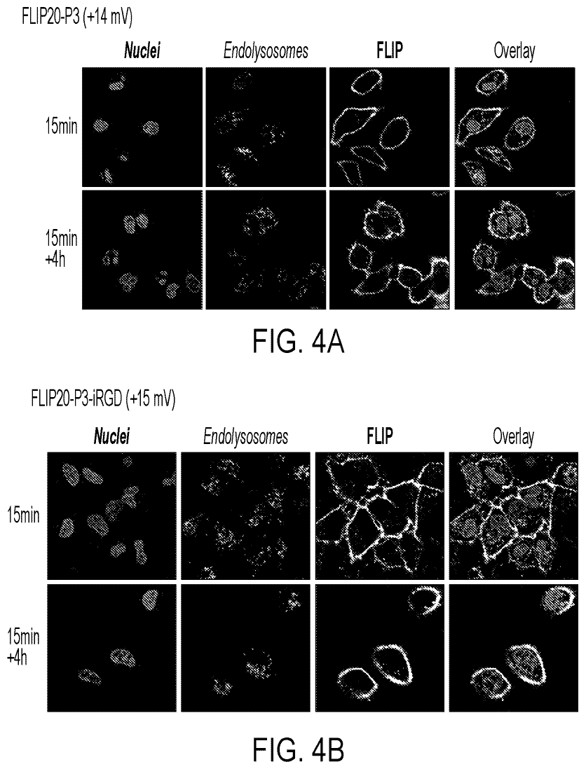

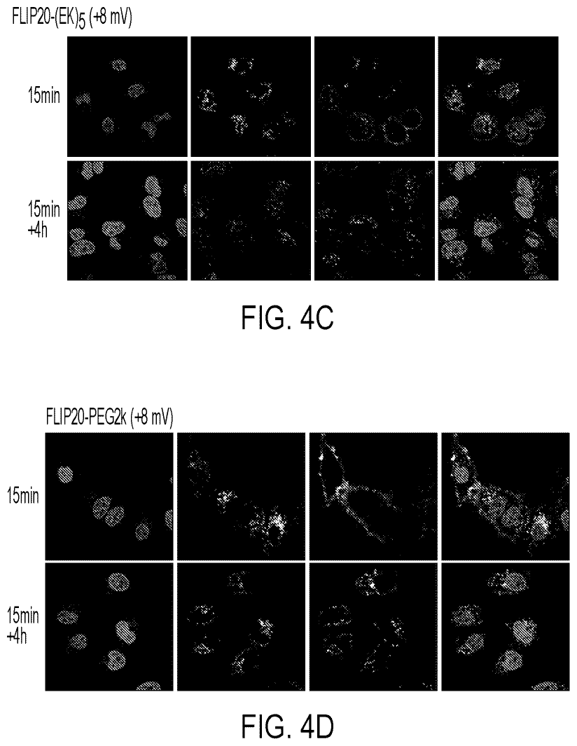

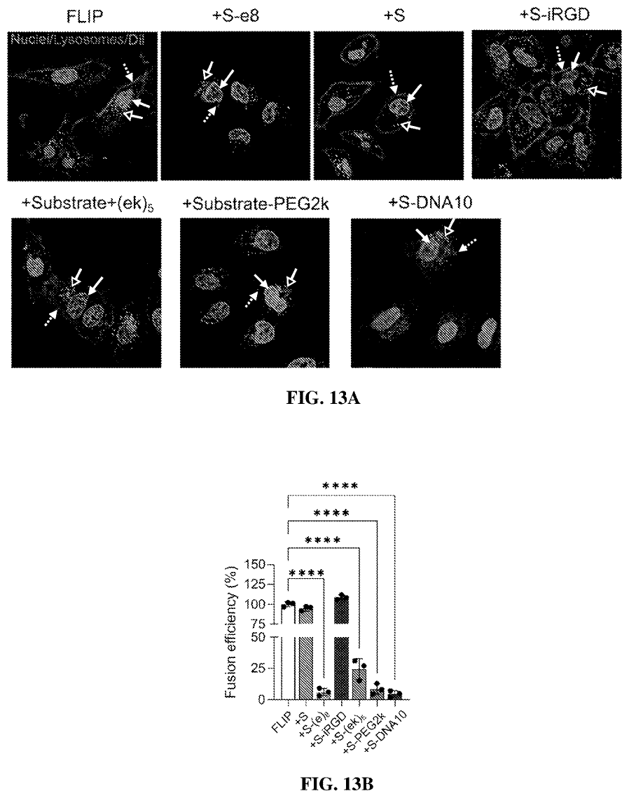

Additional candidates were tested as potential fusion inhibitors. Peptides such as zwitterionic chains (e.g., I7 in Table 2), synthetic polymers such as PEG2000 (10% on surface groups; I5 in Table 2), and short single-stranded oligonucleotides (e.g., ssDNA 10-mer [DNA10], 18 in Table 2) were also effective in blocking the fusogenicity of the FLIPs (FIGS. 4A, 4C, 4D, and 13A-13B). Conversely, a peptide termed iRGD (a tumor homing motif with the sequence CRGDKGPDC (SEQ ID NO: 2), wherein each cysteine (C) serves as a bridging amino acid) seemed to promote the fusion of FLIP with plasma membrane (FIGS. 4B and 13A-13B).

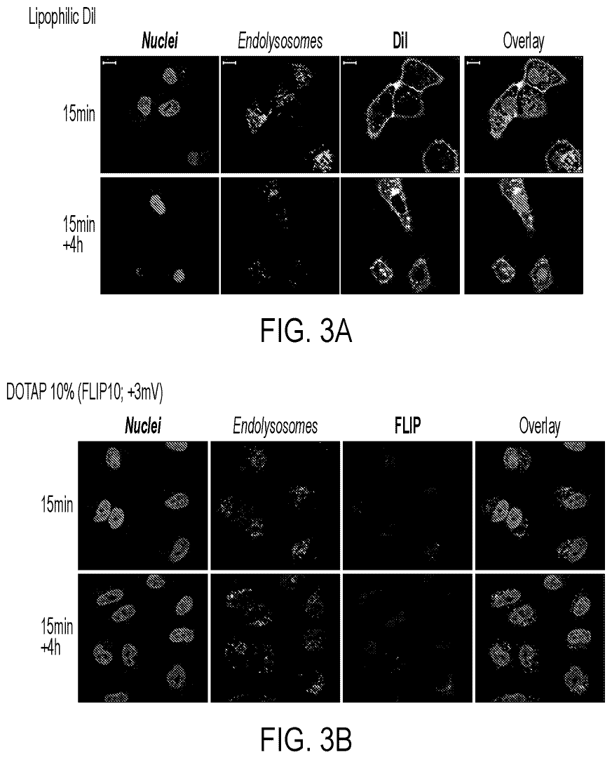

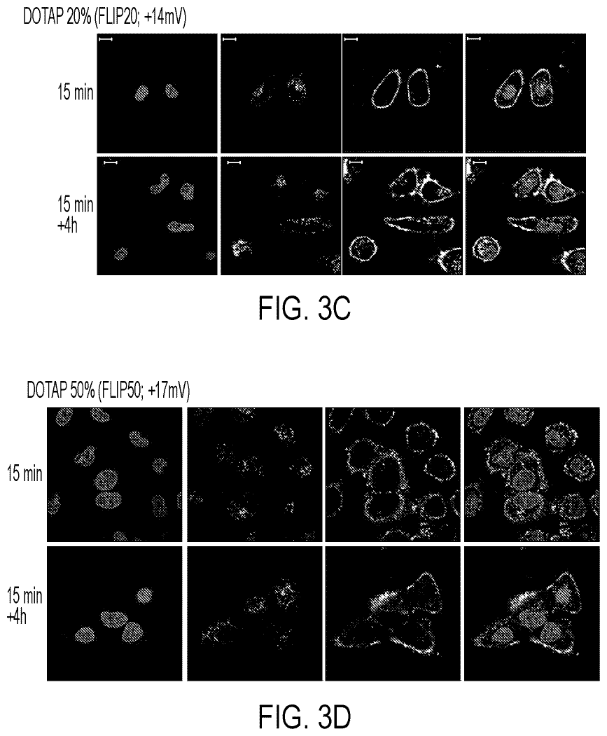

In addition, the physiochemical properties (e.g., rigidity, surface charge, or reactivity) of liposomes were modified by altering the lipid composition of the CFLIPs. The formulation of fusogenic liposomal platforms (FLIPs) included lipids with low phase transition temperature (generally below physiological temperatures). Without being bound by a particular theory, membrane fluidity may help promote fusion. In addition, the overall zeta potential of FLIPs was designed to be moderately positive to promote electrostatic attraction to anionic cell membranes, as it has been observed that moderate positive charges (e.g., +12 mV to +18 mV) maximize the fusogenic property of the liposomes (Kim, et al. “Securing the Payload, Finding the Cell, and Avoiding the Endosome: Peptide-Targeted, Fusogenic Porous Silicon Nanoparticles for Delivery of siRNA.” Advanced Materials 31, 1902952 (2019)). Therefore, the effect of altering cationic lipid (e.g., DOTAP) ratios on the fusogenicity of CFLIPs was tested. The surface charge of the liposomes increased as the ratio of cationic DOTAP increased, plateauing at 20% or above of the total lipids (FIGS. 3A-3D). Significant fusion was observed between cellular plasma membrane and liposomes with 20% and 50% DOTAP after both short and long incubations (FIGS. 3C and 3D). The CFLIP with 20% DOTAP (CFLIP20) was chosen for further experimentation to minimize in vitro and in vivo toxicity.

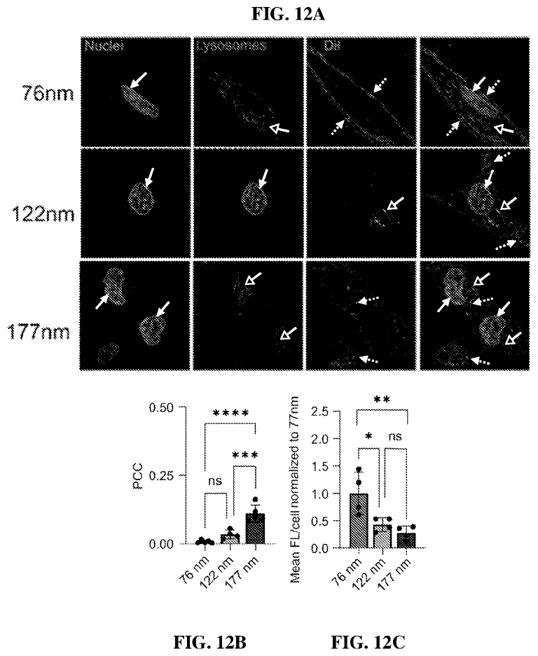

The effect of size on fusogenicity of liposomes was likewise examined. Three FLIPs of different sizes were generated (diameters of 76 nm, 122 nm, and 177 nm). The zeta potentials of the three differently-sized FLIPs were negligibly nearly identical (+12 to +15 mV), ruling out the possibility of differences in fusogenicity caused by surface charges of the particles. The 76 nm FLIP displayed the lowest Pearson correlation coefficient (PCC) (FIGS. 12A-12B), but demonstrated the highest mean fluorescence on plasma membrane (FL) per cell (FIGS. 12A and 12C), indicating a superior interaction with cells via membrane fusion rather than endocytosis. An increased portion of endocytosed liposomes was observed in larger FLIPs as measured via PCC, indicating less efficient fusogenicity (FIG. 12B).

In summary, the fusogenic characteristics of CFLIPs were altered at least in part by varying: a) the ratios between cationic lipids, structural lipids, and fusion inhibitors, b) the size of the liposome, and c) the type of fusion inhibitor used.

Conditions affecting CFLIP shelf-life was also examined. Long-term shelf-life of CFLIP formulations was sought with the goal of granting patients ease-of-access, particularly in low-resource settings. Sugar excipients were used to stabilize liposomal structure during freezing and lyophilization. CFLIPs in 1× phosphate-buffered saline (PBS) with or without sugar excipients were measured for size, polydispersity, and zeta potential prior to being frozen either a) with a gradient drop of temperature at 1° C. per minute until reaching −80° C., or b) directly frozen at −20° C. for 30 minutes, then transferred to −80° C. The frozen CFLIPs were further lyophilized in a benchtop Labconco freeze dryer (−64° C., <10 mbar) overnight. The frozen CFLIPs were then thawed at room temperature for 30 minutes and gently vortexed before being reconstituted in PBS with or without sugar excipients. The thawed, reconstituted CFLIPs were again measured for size, polydispersity, and zeta potential.

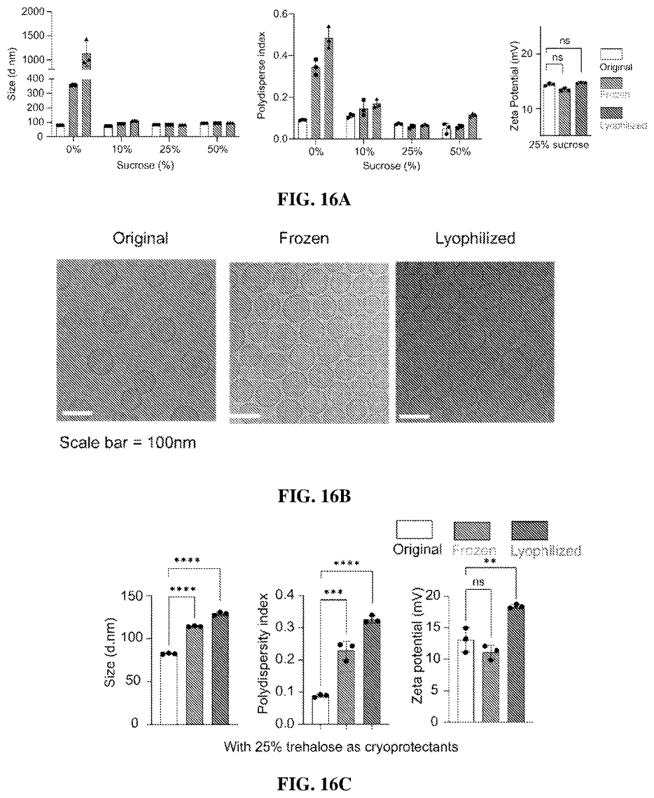

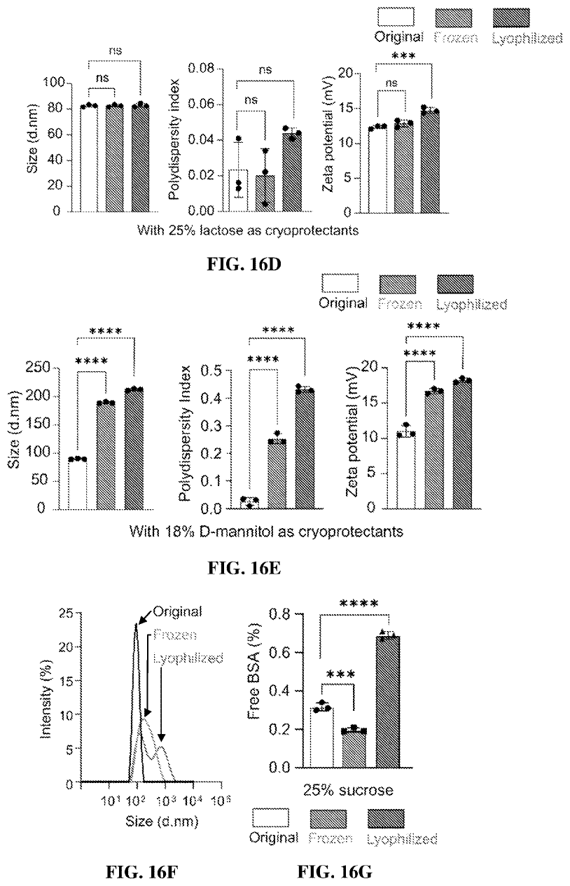

It was determined that between 5% and 25% w/v sucrose and lactose in a buffered solution allowed for full maintenance of structural integrity of the CFLIPs before and after freezing. This was measured by size, polydispersity, and zeta potential (FIGS. 16A-16B and 16D). Lactose and sucrose also helped CFLIPs withstand lyophilization (FIGS. 16A-16B and 16D). Conversely, trehalose (25% w/v) and D-mannitol (18% w/v) exhibited limited utility as structural stabilizers of the CFLIPs during freezing and lyophilization (FIGS. 16C, 16E, and 16F).

### Example 2: Encapsulation of Therapeutic Cargos (e.g., Peptides and Proteins) in CFLIPs and In Vitro Evaluation of Therapeutic Efficacy

As an example of protein cargo, recombinant mouse granzyme B (GzmB) was loaded into an CFLIP activatable by neutrophil elastase (NE) (FIG. 5A). The CFLIP was negatively charged (−15 to −20 mV) and the size was around 50-100 nm. GzmB was labeled with Cy5 and widespread fluorescence was detected in the cytoplasm only when the CFLIPs were pre-incubated with the recombinant NE (FIG. 5B, top row), but not in the cells with the intact CFLIPs (FIG. 5B, bottom row), suggesting the successful cytosolic delivery of GzmB in the presence of the NE activator. Similar cytosolic delivery was also observed in the delivery of a small molecule (calcein) (FIG. 5C) and a Cy5-tagged model protein (bovine serum albumin) (Cy5-BSA) (FIG. 5D).

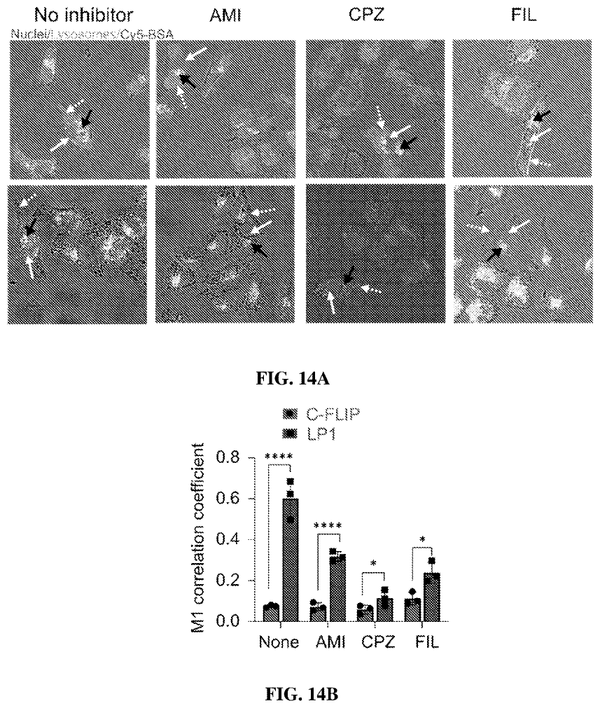

It was further confirmed that the cytosolic delivery of cargo proteins via CFLIPs is independent of endocytosis. Cy5-BSA was delivered to KP lung cancer cells using CFLIPs pre-activated with recombinant NE or non-fusogenic liposomes (FIG. 14A, top row and bottom row, respectively). The cells were incubated for one hour with or without an inhibitors of endocytosis prior to the delivery of Cy5-BSA. The endocytosis inhibitors used (AMI=amilorate; CPZ=chlorpromazine; and FIL=filipin complex III) inhibit micropinocytosis, clathrin-mediated endocytosis, and caveolae-mediated endocytosis, respectively. While the Cy5-BSA delivered via the positively charged non-fusogenic liposomes were was mainly internalized into endosomes via clathrin-mediated endocytosis, none of the endocytosis inhibitors caused a significant difference to the pattern of intracellular delivery, nor the amount of Cy5-BSA delivered when delivered via CFLIP (FIGS. 14A-14B).

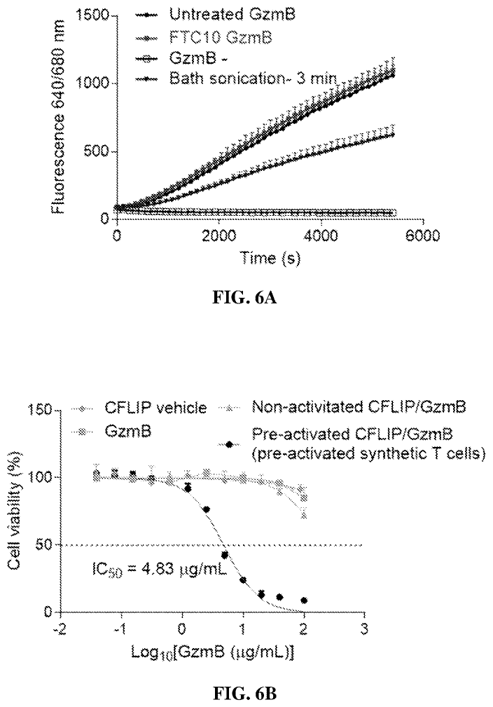

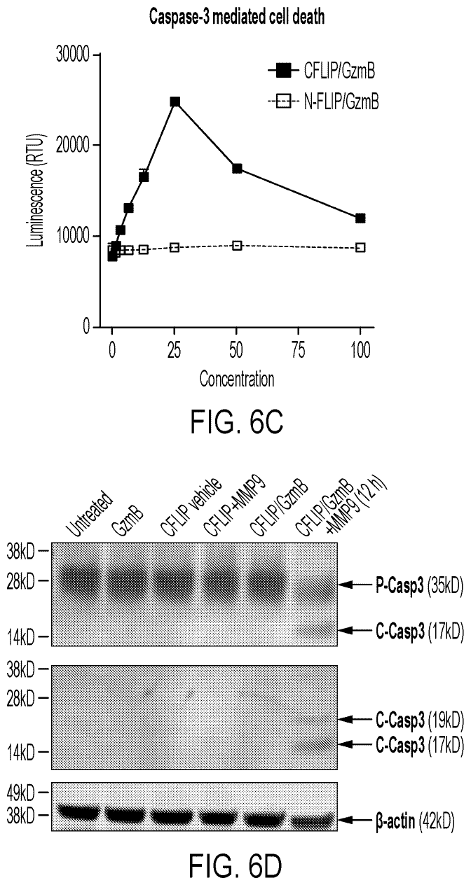

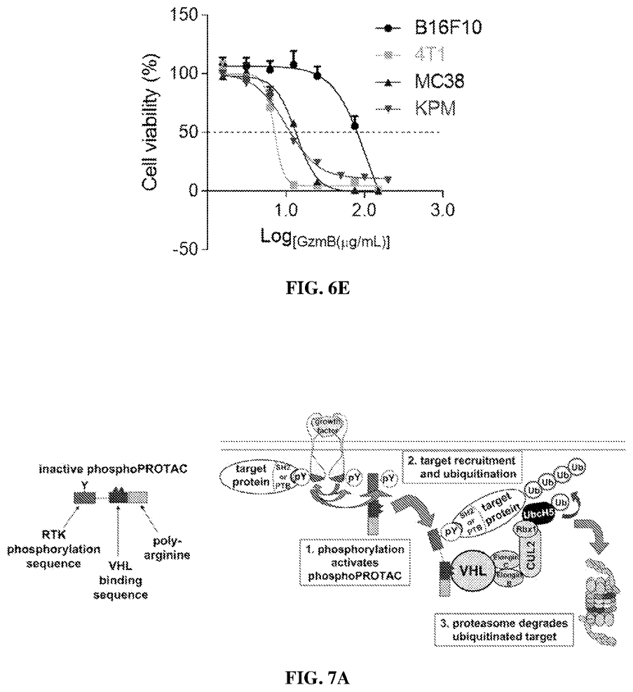

The activity of GzmB-encapsulated CFLIPs (CLFIPs/GzmB) was tested in vitro. GzmB is a cytotoxic enzyme that helps initiates=programmed cell partly by activating caspase 3 in cytosols. Sonication and repeated freeze-thaw cycles were tested as methods to encapsulate cargo in liposomal nanoparticles. GzmB activity was well-preserved with multiple freeze-thaw cycles, but not after sonication (FIG. 6A). The liposomal nanoparticles were then extruded and functionalized with fusion inhibitors and tumor-targeting motifs to form synthetic T cells. Synthetic T cells were evaluated on a KP-derived mouse lung cancer cell line and showed that 1) empty CFLIPs (i.e., liposomal vehicle), 2) naked GzmB, and 3) non-activated CFLIPs/GzmB (i.e., non-activated synthetic T cells), all exhibited no or limited cytotoxicity against the cultured lung cancer cells (FIG. 6B). The cancer cells were then incubated with pre-activated CFLIPs/GzmB (i.e., in vitro incubation of CFLIPs with recombinant MMP9; i.e., pre-activated synthetic T cells) and a significant decrease in cell viability was observed, with IC50=4.8 μg/mL (FIG. 6B). In addition, active caspase 3 was detected intracellularly in the cancer cell only in the group treated with the pre-activated synthetic T cells (FIGS. 6C and 6D). The in vitro antitumor activity of the pre-activated synthetic T cells was observed in other mouse cancer cell lines (FIG. 6E).

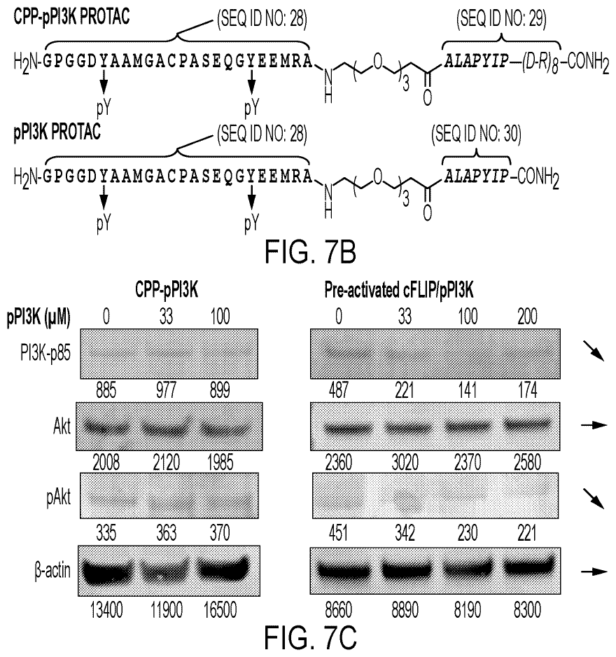

The application of CFLIPs with another model cargo, namely peptide-based proteolysis targeting chimeric (PROTAC) technology, was also tested. Specifically, a PROTAC described by Craig Crews et al. was used.¹¹ The PROTAC in this reference was transported into cells with the aid of a cell-penetrating peptide (CPP) and selectively degraded the catalytic domain (p85) of PI3K upon activation via phosphorylation of receptor tyrosine kinase (RTK) by growth factors. In contrast, in the experiments shown in FIGS. 7A-7C of the present application, the peptide was phosphorylated at two tyrosine sites of the p85 binding domain (FIGS. 7A and 7B; Y-to-pY) in the synthetic step to mimic endogenous phosphorylation. With the use of CPP, phosphorylated PI3K PROTAC (pPROTAC) did not degrade the target protein, which indicated the loss of its ability either to enter the cells or to bind to targeted p85 (FIG. 7B, left column) partly due to the presence of negatively charged phosphorate groups weakening penetrating capability of the CPP. Encapsulation of the phosphorylated peptide (without the CPP domain) in the conditional CFLIPs led to successful degradation of PI3K by approximately 70% at 100 μM equivalent pPI3K in 2D cancer cell culture such as KP lung cancer cells, and significantly decreased the downstream phosphorylation of Akt (FIG. 7C, right column).

### Example 3: In Vivo Delivery of Protease Responsive CFLIPs for Non-Communicable Diseases (e.g., Lung Cancer)

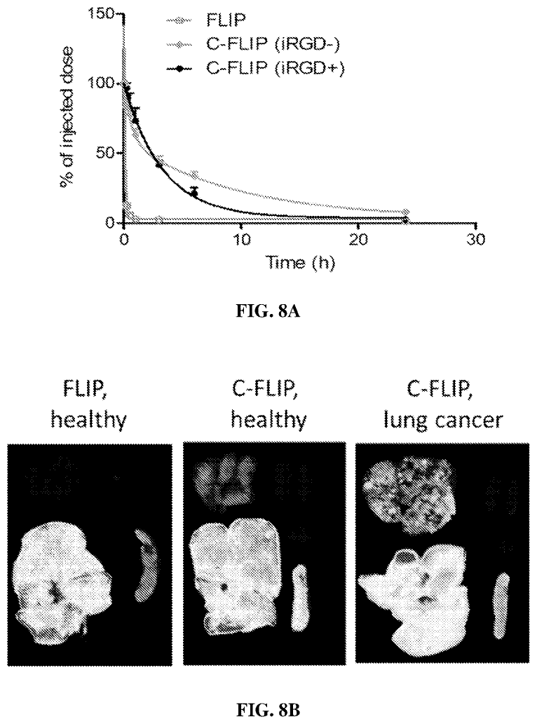

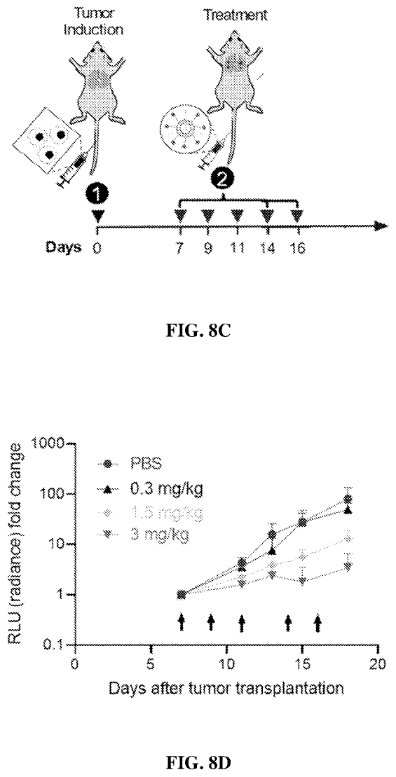

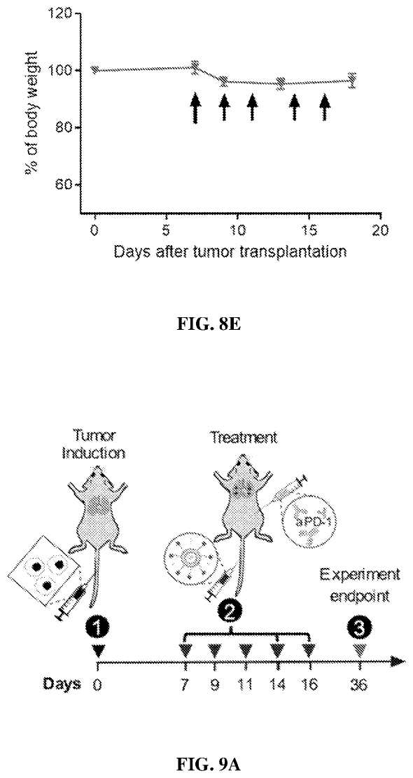

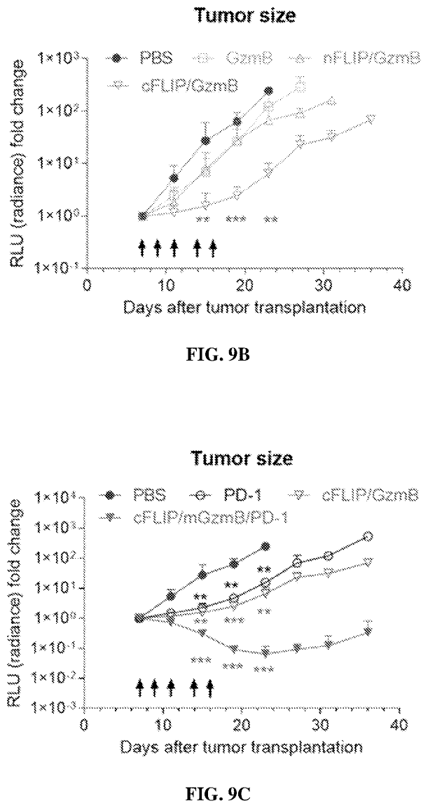

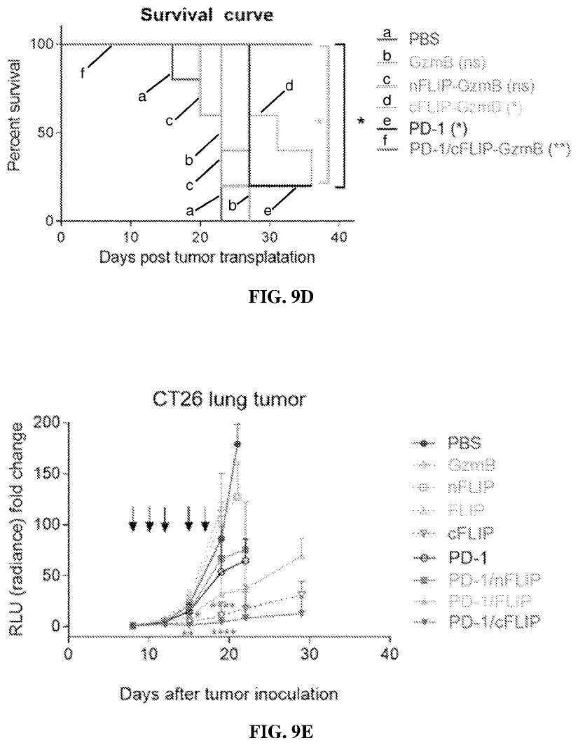

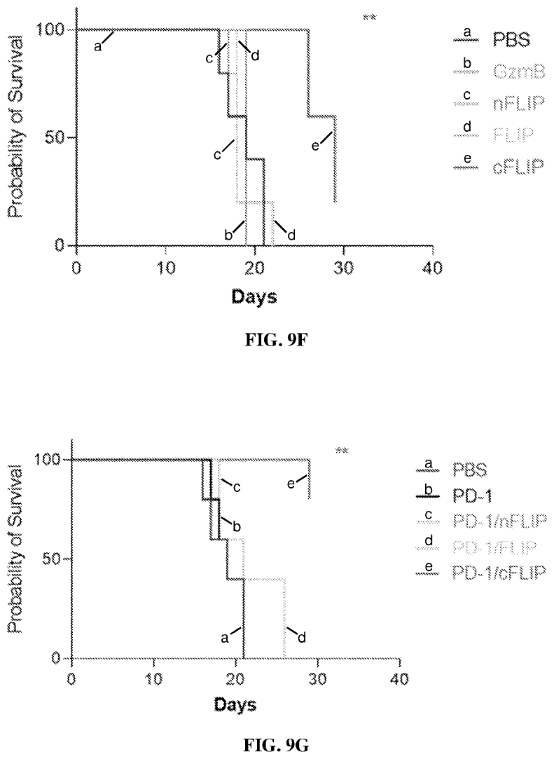

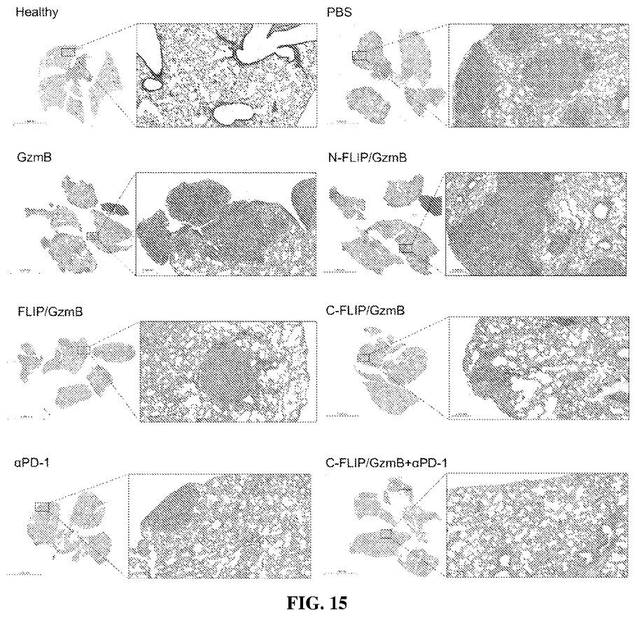

The in vivo performance of the formulated synthetic T cells (i.e., CFLIP/GzmB) was evaluated. As an initial proof of concept, the formulation was tested in an orthotopic lung tumor mouse model induced via intravenous administration of KP cancer cells expressing luciferase that allowed for monitoring tumor growth in vivo. Several proteases (e.g., MMP9, MMP13, and PRSS1) have been shown to be upregulated in lung cancer, which guided the selection of protease-sensitive substrates when designing the synthetic T cells (Hynes and Naba, “Overview of the matrisome—An inventory of extracellular matrix constituents and functions.” Cold Spring Harbor Perspectives in Biology, 4 (2012); Naba et al., “The matrisome: In silico definition an in vivo characterization by proteomics of normal and tumor extracellular matrices.” Molecular and Cellular Proteomics, 11 (2012)). Shielding FLIPs with negative fusion inhibitors (13 in Table 2) significantly prolonged circulation time and improved accumulation in the lungs in both healthy and tumor-bearing mice (FIGS. 8A and 8B). The therapeutic efficacy of the synthetic T cells with activatable fusogenicity was also evaluated. Over 5 doses of treatment (FIG. 8C), the synthetic T cells as a monotherapy showed significant tumor growth suppression with each dose up to 3 mg GzmB/kg body weight (FIG. 8D) without the loss of body weight (FIG. 8E), 3 mg/kg was thus chosen for the following efficacy test in vivo. In comparison with non-activatable FLIPs (NFLIPs) encapsulating GzmB (i.e., non-activatable synthetic T cells) (nFLIP-GzmB) and delivery of vehicle-free GzmB (GzmB), activatable synthetic T cells significantly inhibited tumor growth and conferred survival advantages (FIGS. 9B and 9D). In combination with an anti-PD1 immune checkpoint inhibitor (PD-1/cFLIP-GzmB), the efficacy of the synthetic T cells was further enhanced (FIGS. 9C and 9D), outperforming the PD-1 treatment alone (PD-1). Specifically, in vivo imaging system did not detect bioluminescence of tumor nodules in 4 out of 5 mice in the cohort treated the combinatorial regimen, suggesting that the synthetic T cells are highly synergistic with immune checkpoint inhibitors, with 100% survival over the observed period of 36 days (FIG. 9D). This finding was supported by histological evaluation of lungs from these mice. Tumor burden was significantly decreased in mice treated with C-FLIP/GzmB compared to mice treated with PBS, vehicle-free GzmB, or non-activatable FLIPs containing GzmB, and this effect was enhanced with co-treatment with an anti-PD1 immune checkpoint inhibitor (FIG. 15).

Protease signatures are also observed at a tissue level, allowing for the tuning of the CFLIP protease cleavage domain to not only disease states but also specific organs as well (e.g., lung, kidney, and spleen).

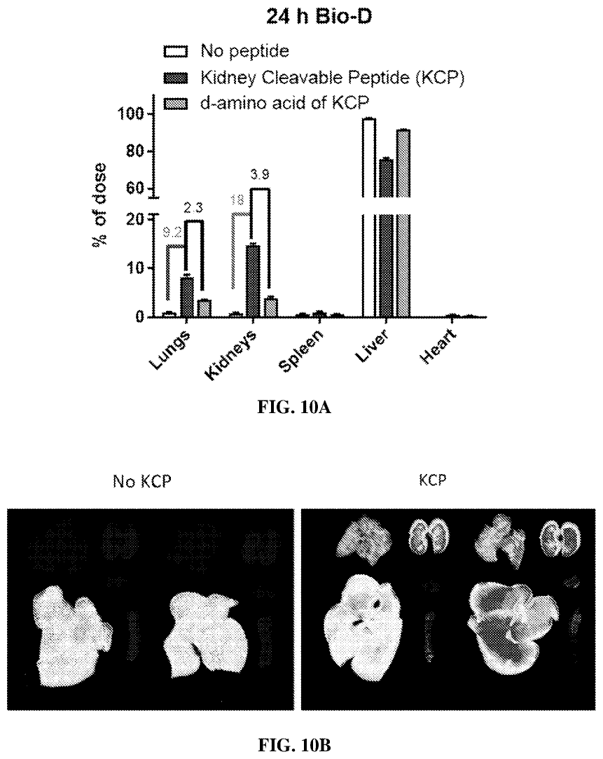

FIGS. 10A-10B shows kidney-specific protease-triggered accumulation of Cy-7-tagged 8-arm polyethylene glycol nanoparticles in the kidneys. The nanoparticle system can accumulate in the kidneys in a substrate-specific manner. Without being bound by a particular theory, kidney-specific proteases may turn charge-neutral nanoparticles to be positively charged nanoparticles by removing anionic peptide sequences, resulting in increased interaction of nanoparticles with renal cells and tissues.

TABLE 7
Lipid, fusion inhibitor, and substrate composition of reagents used in the
Examples and Figures.

| | Lipid composition with | | |
|---|---|---|---|
| | indicated ratio unless | | |
| FIG. | indicated otherwise | Fusion inhibitor | Substrate |
| FIG. 2A | DOTAP/DMPC/DOPE- | eGVndneeGFFsAr (SEQ ID | NE substrate: |
| | MAL/DSPE-MAL is | NO: 1) | Nle(Obzl)-M(O)2- |
| | 20/70/7/3 | | Oic-Abu-C |
| FIGs. 2B | DOTAP/DMPC/DOPE- | eGVndneeGFFsAr (SEQ ID | NE substrate: |
| and 2C | MAL/DSPE-MAL is | NO: 1) | Nle(Obzl)-M(O)2- |
| | 20/70/7/3 | | Oic-Abu-C |
| | | | MMP9-substrate: |
| | | | GGPLGVRGKC |
| | | | (SEQ ID NO: 8) |
| | | | MMP13-substrate: |
| | | | GGPVPLSLVMC |
| | | | (SEQ ID NO: 14) |
| FIG. 2D | N/A | N/A | N/A |
| FIG. 2E | DOTAP/DMPC/DOPE- | eGVndneeGFFsAr (SEQ ID | NE substrate: |
| | MAL/DSPE-MAL is | NO: 1) | Nle(Obzl)-M(O)2- |
| | 20/70/7/3 | | Oic-Abu-C |
| FIG. 3A | N/A | N/A | N/A |
| FIG. 3B | DOTAP/DMPC/DOPE- | N/A | N/A |
| | MAL/DSPE-MAL is | | |
| | 10/80/7/3 | | |
| FIG. 3C | DOTAP/DMPC/DOPE- | N/A | N/A |
| | MAL/DSPE-MAL is | | |
| | 20/70/7/3 | | |
| FIG. 3D | DOTAP/DMPC/DOPE- | N/A | N/A |
| | MAL/DSPE-MAL is | | |
| | 50/40/7/3 | | |
| FIG. 4A | DOTAP/DMPC/DOPE- | N/A | GGPLGVRGKC |
| | MAL/DSPE-MAL is | | (SEQ ID NO: 8) |
| | 20/70/7/3 | | |
| FIG. 4B | DOTAP/DMPC/DOPE- | GGCRGDKGPDC (SEQ ID | GGPLGVRGKC |
| | MAL/DSPE-MAL is | NO: 26) | (SEQ ID NO: 8) |
| | 20/70/7/3 | | |
| FIG. 4C | DOTAP/DMPC/DOPE- | ekekekekek (SEQ ID NO: 7) | GGPLGVRGKC |
| | MAL/DSPE-MAL is | | (SEQ ID NO: 8) |
| | 20/70/7/3 | | |
| FIG. 4D | DOTAP/DMPC/DOPE- | PEG2000 | GGPLGVRGKC |
| | MAL/DSPE-MAL is | | (SEQ ID NO: 8) |
| | 50/40/7/3 | | |
| FIG. 5A | DOTAP/DMPC/DOPE- | eeeeeeeee (SEQ ID NO: 5) | Nle(Obzl)-M(0)2- |
| | MAL/DSPE-MAL is | | Oic-Abu-C |
| | 20/70/7/3 | | |
| FIG. SB | DOTAP/DMPC/DOPE- | eeeeeeeee (SEQ ID NO: 5) | Nle(Obzl)-M(O)2- |
| | MAL/DSPE-MAL is | | Oic-Abu-C |
| | 20/70/7/3 | | |
| FIG. 5C | DOTAP/DMPC/DOPE- | eeceecece (SEQ ID NO: 5) | Nle(Obzl)-M(O)2- |
| | MAL/DSPE-MAL is | | Oic-Abu-C |
| | 20/70/7/3 | | |
| FIG. 5D | DOTAP/DMPC/DOPE- | ececeecee (SEQ ID NO: 5) | GGPLGVRGKC |
| | MAL/DSPE-MAL is | | (SEQ ID NO: 8) |
| | 50/40/7/3 | | |
| FIG. 6A | N/A | N/A | N/A |
| FIG. 6B | DOTAP/DMPC/DOPE- | ceceeeeee (SEQ ID NO: 5) | GGPLGVRGKC |
| | MAL/DSPE-MAL is | | (SEQ ID NO: 8) |
| | 20/70/7/3 | | |
| FIG. 6C | DOTAP/DMPC/DOPE- | eceececee (SEQ ID NO: 5) | GGPLGVRGKC |
| | MAL/DSPE-MAL is | | (SEQ ID NO: 8) |
| | 20/70/7/3 | | |
| FIG. 6D | DOTAP/DMPC/DOPE- | eeeeeeeee (SEQ ID NO: 5) | GGPLGVRGKC |
| | MAL/DSPE-MAL is | | (SEQ ID NO: 8) |
| | 20/70/7/3 | | |
| FIG. 6E | DOTAP/DMPC/DOPE- | eeeeeeeee (SEQ ID NO: 5) | GGPLGVRGKC |
| | MAL/DSPE-MAL is | | (SEQ ID NO: 8) |
| | 20/70/7/3 | | |
| FIGs. 7A | N/A | N/A | N/A |
| and 7B | | | |
| FIG. 70 | DOTAP/DMPC/DOPE- | eeeeeeeee (SEQ ID NO: 5) | GGPLGVRGKC |
| | MAL/DSPE-MAL is | | (SEQ ID NO: 8) |
| | 20/70/7/3 | | |
| FIGs. 8A- | DOTAP/DMPC/DOPE- | ececeecee (SEQ ID NO: 5) | GGPLGVRGKC |
| 8E and | MAL/DSPE-MAL is | | (SEQ ID NO: 8) |
| FIGs. 9A- | 20/70/7/3 | | |
| 9G | | | |
| FIGs. 8A- | DOTAP/DMPC/DOPE- | eeeeeeeee (SEQ ID NO: 5) | GGPLGVRGKC |
| 8E and | MAL/DSPE-MAL is | | (SEQ ID NO: 8) |
| FIGs. 9A- | 20/70/7/3 | | GNSGRAVTYGC |
| 9G | | | (SEQ ID NO: 18) |
| FIGs. | 8-arm polyethylene glycol** | N/A | GNSGRAVTYGC |
| 10A and | | | (SEQ ID NO: 18) |
| 10B | | | |
| FIG. 11 | DOTAP/DMPC/DOPE- | eceececee (SEQ ID NO: 5) | GGPLGVRGKC |
| | MAL/DSPE-MAL is | | (SEQ ID NO: 8) |
| | 20/70/7/3 | | |
| FIGs. | DOTAP/DMPC/DOPEMAL/ | N/A | N/A |
| 12A and | DSPE-MAL is | | |
| 12B | 20/70/7/3 | | |
| FIGs. | DOTAP/DMPC/DOPEMAL/ | eceecece (SEQ ID NO: 4); | GGPLGVRGKC |
| 13A and | DSPE-MAL is | PEG2000; | (SEQ ID NO: 8) |
| 13B | 20/70/7/3 | ekekekekek (SEQ ID NO: | |
| | | 7); | |
| | | TCATAGTTAG/3Thiol | |
| | | (SEQ ID NO: 27); | |
| | | CGGCRGDKGPDC (SEQ | |
| | | ID NO: 23) (C2&C3 bridge) | |
| FIGs. | DOTAP/DMPC/DOPEMAL/ | ceceecee (SEQ ID NO: 4) | GGPLGVRGKC |
| 14A and | DSPE-MAL is | | (SEQ ID NO: 8) |
| 14B | 20/70/7/3 | | |
| FIG. 15 | DOTAP/DMPC/DOPEMAL/ | eeeeeece (SEQ ID NO: 4) | GGPLGVRGKC |
| | DSPE-MAL is | | (SEQ ID NO: 8) |
| | 20/70/7/3 | | |
| FIGs. | DOTAP/DMPC/DOPEMAL/ | eeeeeeee (SEQ ID NO: 4) | GGPLGVRGKC |
| 16A-16G | DSPE-MAL is | | (SEQ ID NO: 8) |
| | 20/70/7/3 | | |

*N/A indicates non-applicable .
**8-arm polyethylene glycol is used as the carrier domain and is not a lipid.

### REFERENCES

1. Gu, L., Deng, Z. J., Roy, S. & Hammond, P. T. A combination RNAi-chemotherapy layer-by-layer nanoparticle for systemic targeting of KRAS/P53 with cisplatin to treat non-small cell lung cancer. Clinical Cancer Research 23, 7312-7323 (2017).
2. Schroeder, A., Levins, C. G., Cortez, C., Langer, R. & Anderson, D. G. Lipid-based nanotherapeutics for siRNA delivery, in Journal of Internal Medicine vol. 267 9-21 (NIH Public Access, 2010).
3. Dudani, J. S., Warren, A. D. & Bhatia, S. N. Harnessing Protease Activity to Improve Cancer Care. Annu. Rev. Cancer Biol 2, 353-76 (2018).
4. Hanahan, D. & Weinberg, R. A. Hallmarks of cancer: The next generation. Cell vol. 144 646-674 (2011).
5. Kwong, G. A. et al. Mass-encoded synthetic biomarkers for multiplexed urinary monitoring of disease. (2012) doi: 10.1038/nbt.2464.
6. Warren, A. D., Kwong, G. A., Wood, D. K., Lin, K. Y. & Bhatia. S. N. Point-of-care diagnostics for noncommunicable diseases using synthetic urinary biomarkers and paper microfluidics. Proc Natl Acad Sci USA 111, 3671-3676 (2014).
7. Lewis, J. G. et al. A serum-resistant cytofectin for cellular delivery of antisense oligodeoxynucleotides and plasmid DNA. Proc Natl Acad Sci USA 93, 3176-3181 (1996).
8. Torchilin, V. P. Recent advances with liposomes as pharmaceutical carriers. Nature Reviews Drug Discovery 4, 145-160 (2005).
9. Balazs, D. A. & Godbey, WT. Liposomes for Use in Gene Delivery. Journal of Drug Delivery 2011, 1-12 (2011).
10. Kim, B. et al. Securing the Payload, Finding the Cell, and Avoiding the Endosome: Peptide-Targeted, Fusogenic Porous Silicon Nanoparticles for Delivery of siRNA. Advanced Materials 31, 1902952 (2019).
11. Hines, J., Gough, J. D., Corson, T. W. & Crews, C. M. Posttranslational protein knockdown coupled to receptor tyrosine kinase activation with phosphoPROTACs. Proc Natl Acad Sci USA 110, 8942-8947 (2013).
12. Hynes, R. O. & Naba, A. Overview of the matrisome—An inventory of extracellular matrix constituents and functions. Cold Spring Harbor Perspectives in Biology 4, (2012).
13. Naba, A. et al. The matrisome: In silico definition and in vivo characterization by proteomics of normal and tumor extracellular matrices. Molecular and Cellular Proteomics 11, (2012).
14. Jiang, L. et al. Direct Tumor Killing and Immunotherapy through Anti-SerpinB9 Therapy. Cell 183, 1219-1233.e18 (2020).
15. Kirkpatrick, J. D. et al. Protease activity sensors enable real-time treatment response monitoring in lymphangioleiomyomatosis. European Respiratory Journal 59, 2100664 (2022).
16. Guidotti, G., Brambilla. L. & Rossi, D. Cell-Penetrating Peptides: From Basic Research to Clinics, Trends in Pharmacological Sciences 38, 406-424 (2017).
17. Banskota, S. et al. Engineered virus-like particles for efficient in vivo delivery of therapeutic proteins. Cell 185, 250-265.e16 (2022).
18. Sun, Y. et al. Phase-separating peptides for direct cytosolic delivery and redox-activated release of macromolecular therapeutics. doi:10.1038/41557-021-00854-4.
19. Tietz, O., Cortezon-Tamarit, F., Chalk, R., Able, S. & Vallis, K. A. Tricyclic cell-penetrating peptides for efficient delivery of functional antibodies into cancer cells. doi:10.1038/s41557-021-00866-0.
20. U.S. patent Ser. No. 10/702,474

The foregoing written specification is considered to be sufficient to enable one skilled in the art to practice the invention. The present invention is not limited in scope by the examples provided, since the examples are intended as illustrations of various aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

1. A reagent comprising: (i) a liposome comprising a cationic lipid that is positively charged at a pH less than 9; (ii) a fusion inhibitor linked to the liposome via (iii) an environmentally-responsive linker, wherein the fusion inhibitor comprises:
(a) nucleic acid, protein, and/or synthetic polymer, optionally wherein the nucleic acid has an overall negative net charge, optionally wherein the nucleic acid fusion inhibitor is 5-30 nucleotides in length, optionally comprising one or more modified nucleotides;
(b) a peptide comprising one or more negatively charged amino acids; and/or
(c) a zwitterionic molecule,
and wherein the liposome is positively charged in the absence of the fusion inhibitor, optionally wherein the environmentally-responsive linker is a protease substrate, optionally wherein the liposome comprises a lipid linked to the fusion inhibitor, optionally wherein the protein is an antibody, and optionally wherein the fusion inhibitor comprises (b) and/or (c).

2. The reagent of claim 1, wherein the liposome comprises one or more lipids selected from the group consisting of 1,2-dioleoyl-3-trimethylammonium-propane (DOTAP), 1,2-dioleoyl-3-dimethylammonium propane (DODAP), 1,2-dimyristoyl-sn-glycero-3-phosphocholine (DMPC), 1,2-dimyristoyl-sn-glycero-3-phosphoethanolamine (DMPE), cholesterol, 1,2-dioleoyl-sn-glycero-3-phosphoethanolamine (DOPE), 1,2-dioleoyl-sn-glycero-3-phosphoethanolamine maleimide (DOPE-MAL), 1,2-distearoyl-sn-glycero-3-phosphoethanolamine maleimide (DSPE-MAL), 1,2-distearoyl-sn-glycero-3-phosphoethanolamine-N-[amino (polyethylene glycol)-2000] maleimide (DSPE-PEG2k-MAL), 1,2-distearoyl-sn-glycero-3-phosphoethanolamine-N-[amino(polyethylene glycol)-2000] DBCO (DSPE-PEG2k-DBCO), 1,2-dimyristoyl-rac-glycero-3-methoxypolyethylene glycol-2000 maleimide (DMG-PEG2k-MAL), C14PEG1k, C14PEG2k, DSPE-PEG2K, DMG-PEG1k, and DMG-PEK2k.

3. The reagent of claim 1 or 2 further comprising a ligand capable of binding to a cell.

4. The reagent of claim 3, wherein the ligand comprises a peptide, ligand, lectin, antibody, and/or nucleic acid molecule, optionally wherein the ligand is a tumor-penetrating ligand and optionally wherein the antibody is a nanobody.

5. The reagent of claim 3 or 4, wherein the ligand is an iRGD peptide, optionally wherein the iRGD peptide comprises the amino acid sequence CRGDKGPDC (SEQ ID NO: 2), in which the two cysteines form a disulfide bridge.

6. The reagent of any one of claims 1-5, wherein the fusion inhibitor comprises a glutamate residue and/or the protease substrate is a cell-specific substrate, tissue-specific substrate, organ-specific substrate, and/or a disease-specific substrate.

7. The reagent of any one of claims 1-6, wherein the fusion inhibitor comprises overall net charge of inhibitor peptides is-3 at physiological pH, optionally wherein the physiological pH is pH 7.4 and optionally wherein the fusion inhibitor comprises at least three negatively charged amino acids.

8. The reagent of any one of claims 1-7, wherein the fusion inhibitor is the peptide of (b) and less than 25% of the peptide is hydrophobic amino acid residues and/or the zwitterionic molecule is a peptide.

9. The reagent of any one of claims 6-8, wherein the fusion inhibitor comprises a lysine or arginine residue.

10. The reagent of any one of claims 1-9, wherein the liposome comprises more than 5% but less than 50% DOTAP.

11. The reagent of any one of claims 1-10, wherein the fusion inhibitor is 5-25 amino acids in length, optionally wherein the fusion inhibitor is 8-17 amino acids in length.

12. The reagent of any one of claims 1-11, comprising: DOTAP, DMPC, DOPE-MAL, and DSPE-PEG2k-MAL.

13. The reagent of claim 12, wherein the ratio of DOTAP/DMPC/DOPE-MAL/DSPE-PEG2k-MAL is 20/70/7/3.

14. The reagent of any one of claims 1-13, wherein the peptide comprises the amino acid sequence:
(a)
(SEQ ID NO: 1)
eGVndneeGFFsAr;
(b)
(SEQ ID NO: 3)
eeceGVndneeGFFsAr;
(c)
(SEQ ID NO: 4)
eeeeeeee;
(d)
(SEQ ID NO: 5)
eeeeeeeee;
(e)
(SEQ ID NO: 6)
kkeeekkeeekkeeek;
and/or
(f)
(SEQ ID NO: 7)
ekekekekek,
wherein a lower case letter in an amino acid sequence indicates a D-isomer amino acid.

15. A composition comprising the reagent of any one of claims 1-11, wherein the liposome encapsulates cargo, optionally wherein the cargo is a therapeutic molecule, optionally wherein the fusion inhibitor is a therapeutic molecule, optionally wherein the therapeutic molecule that is the fusion inhibitor and/or the cargo is a therapeutic nucleic acid, a therapeutic peptide, or a therapeutic protein, optionally wherein the cargo is a molecule that is greater than 1 kDa in size.

16. The composition of claim 15 further comprising a pharmaceutically acceptable excipient, optionally wherein the pharmaceutically acceptable excipient is sucrose or lactose, optionally wherein the composition comprises the reagent in a solution comprising the pharmaceutically acceptable excipient, optionally wherein the solution comprises between 5% to 25% weight per volume (w/v) of the pharmaceutically acceptable excipient.

17. The composition of claim 15, wherein the therapeutic molecule is capable of inducing cell death in a target cell and/or wherein the therapeutic molecule is granzyme B or phosphorylated PI3K proteolysis targeting chimeric molecule (pPROTAC).

18. A method comprising administering the composition of claim 15 or 16 to a subject, optionally wherein the method comprises detecting the fusion inhibitor in a sample from the subject, optionally wherein the sample is urine, optionally wherein the fusion inhibitor targets the reagent to a cell, tissue, and/or organ in the subject.

19. The method of claim 18, wherein:
(a) the subject has cancer, optionally wherein the cancer is lung cancer;
(b) the reagent localizes to a cell, tissue, and/or organ more than another cell, tissue, and/or organ;
(c) delivery of the cargo to a cell, tissue, or organ expressing a protease is increased relative to a cell, tissue, and/or organ with reduced expression of the protease; and/or
(d) the method delivers a therapeutic molecule capable of inducing cell death in a target cell.

20. The method of claim 19, wherein the method reduces the size of a tumor in the subject.
